(19) United States
(12) Patent Application Publication
(10) Pub. No.: US 2017/0231931 A1
(43) Pub. Date: Aug. 17, 2017

(54) PRODUCTS FOR THE TREATMENT AND PREVENTION OF NEUROLOGICAL DISORDERS COURSING WITH A COGNITION DEFICIT OR IMPAIRMENT, AND OF NEURODEGENERATIVE DISEASES

(71) Applicant: FUNDACIÓN PARA LA INVESTIGACIÓN MÉDICA APLICADA, Pamplona (ES)

(72) Inventors: María del Mar CUADRADO TEJEDOR, Pamplona (ES); Ana María GARCÍA OSTA, Pamplona (ES); Julen OYARZABAL SANTAMARINA, Pamplona (ES); Maria Obdulia RABAL GRACIA, Pamplona (ES)

(21) Appl. No.: 15/504,281

(22) PCT Filed: Aug. 25, 2015
(86) PCT No.: PCT/EP2015/069392

§ 371 Date: Feb. 15, 2017

(30) Foreign Application Priority Data
Aug. 26, 2014 (EP) 14382326.8

(51) Int. Cl. A61K 31/167; A61K 31/519; A61K 31/4985; A61K 31/53

(52) U.S. Cl. CPC A61K 31/167; A61K 31/53; A61K 31/519; A61K 31/4985

(57) ABSTRACT

The invention relates to a product that comprises a compound of formula (1-01) to (1-47) and a compound of formula (2-01) to (2-26), or their pharmaceutically or veterinary acceptable salts thereof, or any stereoisomers either of the compounds or of any of their pharmaceutically or veterinary acceptable salts. The invention also relates to said product for use in the treatment and/or prevention of a neurological disorder coursing with a cognition deficit or impairment, or a neurodegenerative disease.

### FIELD OF THE INVENTION

The present invention is encompassed in the pharmaceutical sector and relates to products comprising a known PDE inhibitor compound and known HDAC inhibitor compound, and to said products for use in the treatment and/or prevention of a neurological disorder coursing with a cognition deficit or impairment, or a neurodegenerative disease.

### BACKGROUND ART

Mild cognitive impairment is characterized by deficits in memory, language and/or other essential cognitive functions that do not interfere with an individual's daily life. The condition often evolves towards dementia, which is characterized by a global deterioration of cognitive abilities to an extent that does interfere with daily life. Alzheimer's disease (AD) is the most common form of dementia among older people and refers to dementia that does not have an antecedent cause, such as stroke, brain trauma, or alcohol; it is characterized by the presence in the brain of extracellular amyloid plaques and intracellular neurofibrillary tangles that provoke neuronal dysfunction and cell death. The increasing number of AD patients associated with the aging of the population makes the development of new disease management/treatment strategies critical.

The search for effective AD management has been largely based on the amyloid (AB) hypothesis, mainly focusing on reducing the number of senile plaques, although with little success to date. Focus is placed now on other hallmarks of the disease such as hyperphosphorylation of the cytoskeletal protein tau, which is the main component of neurofibrillary tangles.

An increase in histone (H3 and/or H4) acetylation using histone deacetylase (HDAC) inhibitors induces chromatin re-structuring, which is associated with gene transcription activation. HDAC proteins are classified in four families: class I (HDAC 1-3, HDAC8), class IIa (HDAC 4, 5, 7 and 9), class I % (HDAC 6 and 10), and class IV (HDAC 11). The expression pattern of each HDAC in the central nervous system (CNS) and its contribution in memory function varies among each subtype.

4-phenylbutyrate (PBA), a HDAC inhibitor, is an effective cognitive-enhancer in the Tg2576 transgenic mouse model of AD, which overexpresses a mutant form of the amyloid precursor protein (APP). Additionally, PBA reversed the pathological hallmarks of the disease and restored dendritic spine loss in this animal model. Taking into account that PBA inhibits class I and IIb HDACs, all these data strongly suggest the potential for therapeutic benefits of HDAC inhibitors in AD, especially for class I HDACs and HDAC6. Class I HDAC inhibitors enhance memory function by increasing histone acetylation levels, which facilitates gene transcription in the brain. Moreover, HDAC6 inhibitors induce tubuline acetylation (AcTub) that may help cytoskeleton stability and protein traffic. This could play an important role in misfolding protein disorders, such as AD, in which HDAC6 inhibitors have been shown to reduce amyloid precursor protein (APP) processing by reducing its amyloid precursor (C99) production.

Moreover, aging is associated with an increase in phosphodiesterase (PDE) expression and activity. Thus, phosphodiesterases (PDEs) are good candidates for non-amyloid targets in cognition deficits in general and in AD in particular. Rolipram, which is a specific PDE4 inhibitor, was the first that proved useful in restoring cognition deficits in animal models of AD. Specific phosphodiesterase (PDE) inhibitors (e.g. PDE5 inhibitors: Sildenafil, or Tadalafil; and, PDE9 inhibitor: PF-4447943 (6-[(3S,4S)-4-methyl-1-(pyrimidin-2-ylmethyl)pyrrolidin-3-yl]-1-(tetrahydro-2H-pyran-4-yl)-1H-pyrazolo[3,4-d]pyrimidin-4(5H)-one)) have been shown to improve memory performance or/and enhance synaptic plasticity and cognitive function in different animal models of AD. PDE inhibitors regulate signaling pathways by elevating levels of cAMP and/or cGMP, which may ultimately promote gene transcription by directly and/or indirectly activating the cAMP response element-binding (CREB). CREB-dependent gene expression underlies long-term memory formation and persistent long-term potentiation (LTP), which are indicators of synaptic plasticity and strength. In the hippocampus, this probably occurs through the formation of new synaptic connections, which are needed to restore cognitive deficits. Thus, by activating the CREB signalling pathway, PDE inhibitors may ameliorate AD symptoms. Moreover, other CREB-independent mechanisms seem to act in synergy to restore cognitive impairment in AD via increase of cAMP and/or cGMP levels. Cognitive performance may be also improved indirectly by means of PDE-inhibitor-mediated increase of cerebral blood flow and/or of brain glucose consumption.

Besides amyloid burden, Tau phosphorylation is another histopathological marker of AD progression. Importantly, it has been shown that the PDE5 inhibitors Sildenafil and Tadalafil, reduce Tau phosphorylation (pTau levels) in different animal models of AD.

Sabayan et al. (The International journal of neuroscience. 2010 vol. 120 (12), pp. 746-51) reviewed PDE-5 inhibitor compounds as novel disease-modifying agents against AD. WO 2012/171974 A1, 20 Dec. 2012, discloses the use of the PDE5 inhibitor compound, Tadalafil, in the prevention or treatment of dementia, particularly, of AD. M. Cuadrado-Tejedor et al. (British Journal of Pharmacology. 2011 vol. 164 (8), pp. 2029-2041) disclosed that the PDE5 inhibitor compound, Sildenafil, improves cognitive impairment in a Tg2576 mouse model of AD. Konsoula et al in a review (Journal of Pharmacological and Toxicological Methods. 2012 vol. 66, pp. 215-220) suggest that treatment with HDAC inhibitor compounds ameliorates neurodegenerative deficiencies and protects against neurodegeneration. Chen et al (British Journal of Pharmacology. 2012 vol. 165 (2), pp. 494-595) demonstrated that the neurotrophic and neuroprotective effects of Pan-HDAC inhibitor compound, Vorinostat, could provide a new therapeutic approach to the treatment of neurodegenerative diseases.

There is still a need of developing products which show improved activity in the treatment and/or prevention of neurological disorders coursing with a cognition deficit or impairment, or neurodegenerative diseases.

### SUMMARY OF THE INVENTION

The inventors have observed a synergistic effect on the epigenetic mark (AcH3) using in vitro assays where Phosphodiesterase (PDE) and Histone deacetylase (HDAC) inhibitor compounds were combined showing that both pathways may interact (FIG. 1) and converge at histone acetylation level leading to an enhance in gene transcription. This hypothesis was confirmed in an in vivo proof of concept using the combination of the HDAC inhibitor compound of formula (2-01) (Vorinostat), and the PDE5 inhibitor compound of formula (1-30) (Tadalafil) to treat chronically aged-Tg2576 mice (AD mouse model). The combination therapy ameliorated memory impairment in aged-Tg2576, whose cognition was severely affected. Furthermore, a significant reduction in amyloid and Tau pathologies was also observed in the brain of the treated animals. Interestingly, these effects were maintained after a 4-week washout period. As far as the inventors know, a synergistic effect by the inhibition of PDE and HDAC, useful to improve cognition, has not been established in the prior art.

Therefore, in a first aspect the invention relates to a product (hereinafter also referred to as “product of the invention”), that comprises

a) a PDE inhibitor compound selected from the group consisting of compounds of formula (1-01) to (1-47), or a pharmaceutically or veterinary acceptable salt thereof, or any stereoisomer thereof, either of the compound of formula (1-01) to (1-47) or of any of its pharmaceutically or veterinary acceptable salts, and

b) a HDAC inhibitor compound selected from the group consisting of compounds of formula (2-01) to (2-26), or a pharmaceutically or veterinary acceptable salt thereof, or any stereoisomer thereof, either of the compound of formula (2-01) to (2-26) or of any of its pharmaceutically or veterinary acceptable salts.

The product of the invention can be a combination, a combined preparation, a pharmaceutical or veterinary composition, and/or a package or a kit of parts. The product of the invention is suitable for simultaneous, concurrent, separate or sequential use of both inhibitor compounds a) and b) as defined above.

As previously described, the product of the invention combining inhibitors of PDEs and HDACs can be useful to improve cognition. Therefore, another aspect of the invention relates to the product of the invention as defined above, for use as a medicament.

In another aspect, the invention relates to the product of the invention as defined above, for use in the treatment and/or prevention of a neurological disorder coursing with a cognition deficit or impairment, or a neurodegenerative disease.

In another aspect the invention relates to the use of a PDE inhibitor compound selected from the group consisting of compounds of formula (1-01) to (1-47) and a HDAC inhibitor compound selected from the group consisting of compounds of formula (2-01) to (2-26), or of a product of the invention as defined above, for the preparation of a medicament for the treatment and/or prevention of a neurological disorder coursing with a cognition deficit or impairment, or a neurodegenerative disease. This aspect may also be formulated as a method for the treatment and/or prevention of a neurological disorder coursing with a cognition deficit or impairment, or a neurodegenerative disease, which comprises administering to a mammal subject in need thereof, including a human subject, a therapeutically effective amount of
a) a PDE inhibitor compound selected from the group consisting of compounds of formula (1-01) to (1-47), or a pharmaceutically or veterinary acceptable salt thereof, or any stereoisomer thereof, either of the compound of formula (1-01) to (1-47) or of any of its pharmaceutically or veterinary acceptable salts, and of
b) a HDAC inhibitor compound selected from the group consisting of compounds of formula (2-01) to (2-26), or a pharmaceutically or veterinary acceptable salt thereof, or any stereoisomer thereof, either of the compound of formula (2-01) to (2-26) or of any of its pharmaceutically or veterinary acceptable salts;

and one or more pharmaceutical acceptable excipients or carriers. In one embodiment, the treatment comprises the simultaneous, concurrent, separate or sequential administration of the PDE inhibitor compound a) and the HDAC inhibitor compound b) as defined above. In an additional aspect, the invention relates to a PDE inhibitor compound a) selected from the group consisting of compounds of formula (1-01) to (1-47), a pharmaceutically or veterinary acceptable salt thereof, and any stereoisomer thereof, either of the compound of formula (1-01) to (1-47) or of any of its pharmaceutically or veterinary acceptable salts, for simultaneous, concurrent, separate or sequential use in combination with a HDAC inhibitor compound b) selected from the group consisting of compounds of formula (2-01) to (2-26), a pharmaceutically or veterinary acceptable salt thereof, and any stereoisomer thereof, either of the compound of formula (2-01) to (2-26) or of any of its pharmaceutically or veterinary acceptable salts, in the treatment and/or prevention of a neurological disorder coursing with a cognition deficit or impairment, or a neurodegenerative disease; it also relates to a HDAC inhibitor compound b) as defined above, for simultaneous, concurrent, separate or sequential use in combination with a PDE inhibitor compound a) as defined above, in the treatment and/or prevention of a neurological disorder coursing with a cognition deficit or impairment, or a neurodegenerative disease.

In an additional aspect, the invention relates to a PDE inhibitor compound a) as defined above, for the preparation of a medicament for simultaneous, concurrent, separate or sequential use in combination therapy with a HDAC inhibitor compound b) as defined above, in the treatment and/or prevention of a neurological disorder coursing with a cognition deficit or impairment.

In an additional aspect, the invention relates to a HDAC inhibitor compound b), for the preparation of a medicament for simultaneous, concurrent, separate or sequential use in combination therapy with a PDE inhibitor compound a), in the treatment and/or prevention of a neurological disorder coursing with a cognition deficit or impairment.

In a further additional aspect the invention relates to a package or kit of parts comprising

i) a PDE inhibitor compound a) selected from the group consisting of compounds of formula (1-01) to (1-47), a pharmaceutically or veterinary acceptable salt thereof, and any stereoisomer thereof, either of the compound of formula (1-01) to (1-47) or of any of its pharmaceutically or veterinary acceptable salts, together with instructions for simultaneous, concurrent, separate or sequential use in combination with a HDAC inhibitor compound b) selected from the group consisting of compounds of formula (2-01) to (2-26), a pharmaceutically or veterinary acceptable salt thereof, and any stereoisomer thereof, either of the compound of formula (2-01) to (2-26) or of any of its pharmaceutically or veterinary acceptable salts; or

ii) a HDAC inhibitor compound b), together with instructions for simultaneous, concurrent, separate or sequential use in combination with a PDE inhibitor compound a);

for use in the treatment and/or prevention of a neurological disorder coursing with a cognition deficit or impairment, or a neurodegenerative disease.

Simultaneous, concurrent, separate and sequential uses of both PDE inhibitor compound a) and HDAC inhibitor compound b) are all considered as particular embodiments of all uses and methods according to the invention described above and below.

### BRIEF DESCRIPTION OF THE DRAWINGS

FIG. 1. Histone acetylation is increased when inhibiting HDAC or by activating histone acetyltransferase (HAT). PDE inhibition induces CREB phosphorylation. pCREB recruits CBP which activates HAT thereby contributing to increase histone acetylation. [symbol] , inhibition; Δ, activation; Ac, acetylation; P, phosphorylation; PDE, phosphodiesterase; HDAC, Histone deacetylase; CBP, CREB binding protein; HAT, histone acetyl transferase.

FIG. 2. Scheme showing time points of treatment, behavioural tests and sacrifice point. FC, Fear Conditioning; MWM, Morris water maze; R_MWM, reversal MWM.

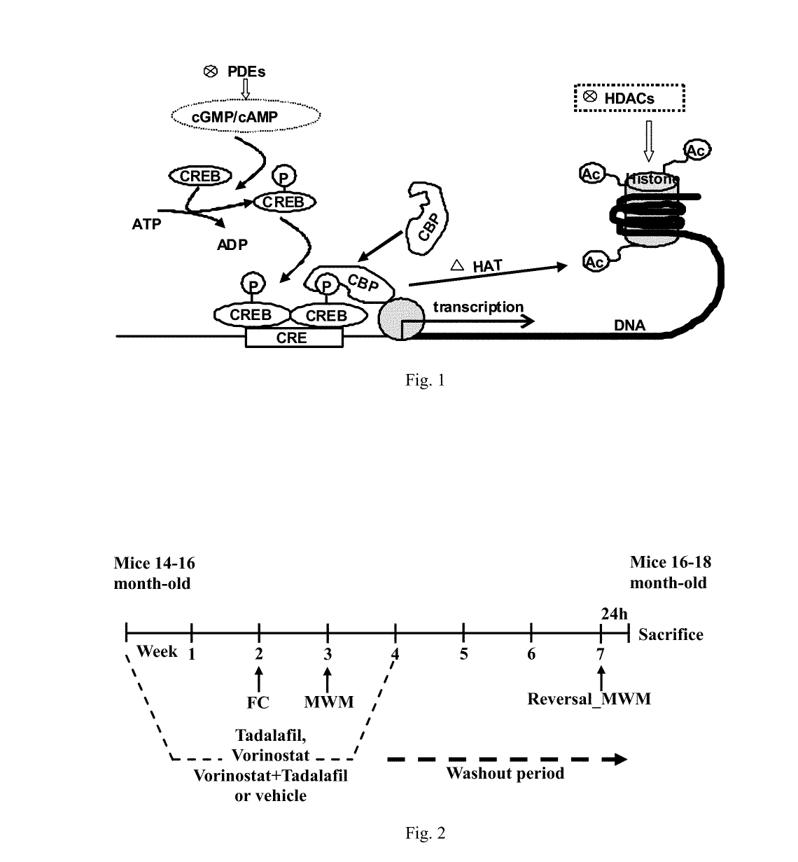

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The term “product” when it is used to refer to the product of the invention shall be understood as any product that comprises a PDE inhibitor compound and a HDAC inhibitor compound, which can be contained within a single composition or formulation, or within in separate compositions or formulations. The product containing the PDE inhibitor compound and the HDAC inhibitor compound forms a functional unity or true combination through a purpose-directed application, in the present case the use for the treatment and/or prevention of a neurological disorder coursing with a cognition deficit or impairment, or a neurodegenerative disease. The product of the invention shall be typically used as a combination or combined preparation for simultaneous, concurrent, separate, or sequential use. Therefore in one embodiment, the product is a combination or a combined preparation.

In one embodiment, optionally in combination with one or more features of the various embodiments described above or below, the product of the invention is a pharmaceutical o veterinary composition that contains both PDE and HDAC inhibitor compounds in the same single composition.

In one embodiment, optionally in combination with one or more features of the various embodiments described above or below, the product of the invention is a package or a kit of parts which contains the PDE inhibitor compound and the HDAC inhibitor compound in single or separate compositions, and is suitable for simultaneous, concurrent, separate or sequential use.

Obviously, any composition or formulation that constitutes or form part of the product of the invention may additionally comprise excipients, adjuvants, and any other convenient pharmaceutical ingredients, including other drugs.

In all embodiments of the invention referring to any compound of formula (1-01) to (1-47) and formula (2-01) to (2-26), the pharmaceutically or veterinary acceptable salts thereof and the stereoisomers either of any of the compounds of formula (1-01) to (1-47) and formula (2-01) to (2-26) or of any of their pharmaceutically or veterinary acceptable salts are always contemplated even if they are not specifically mentioned.

There is no limitation on the type of salt that can be used, provided that these are pharmaceutically or veterinary acceptable when they are used for therapeutic purposes. The term “pharmaceutically or veterinary acceptable salts”, embraces salts commonly used to form alkali metal salts and to form addition salts of free acids or free bases.

The preparation of pharmaceutically or veterinary acceptable salts of the compounds of formula (1-01) to (1-47) and formula (2-01) to (2-26) can be carried out by methods known in the art. For instance, they can be prepared from the parent compound, which contains a basic or acidic moiety, by conventional chemical methods. Generally, such salts are, for example, prepared by reacting the free acid or base forms of these compounds with a stoichiometric amount of the appropriate pharmaceutically or veterinary acceptable base or acid in water or in an organic solvent or in a mixture of them. The compounds of formula (1-01) to (1-47) and formula (2-01) to (2-26) and their salts may differ in some physical properties but they are equivalent for the purposes of the present invention.

Some compounds of formula (1-01) to (1-47) and formula (2-01) to (2-26) may be in crystalline form either as free solvation compounds or as solvates (e.g. hydrates) and it is intended that both forms are within the scope of the present invention. Methods of solvation are generally known within the art. In general, the solvated forms with pharmaceutically or veterinary acceptable solvents such as water, ethanol and the like are equivalent to the unsolvated forms for the purposes of the invention.

Some compounds of formula (1-01) to (1-47) and formula (2-01) to (2-26) can have chiral centers that can give rise to various stereoisomers. As used herein, the term “stereoisomer” refers to all isomers of individual compounds that differ only in the orientation of their atoms in space. The term stereoisomer includes mirror image isomers (enantiomers), mixtures of mirror image isomers (racemates, racemic mixtures), geometric (cis/trans or syn/anti or E/Z) isomers, and isomers of compounds with more than one chiral center that are not mirror images of one another (diastereoisomers). The present invention relates to each of these stereoisomers and also mixtures thereof.

Diastereoisomers and enantiomers can be separated by conventional techniques such as chromatography or fractional crystallization. Optical isomers can be resolved by conventional techniques of optical resolution to give optically pure isomers. This resolution can be carried out on any chiral synthetic intermediate or on compounds of formula (1-01) to (1-47) and formula (2-01) to (2-26). Optically pure isomers can also be individually obtained using enantiospecific synthesis.

The term “inhibitor compound” as used herein refers to the capacity of a compound to inhibit partially or totally, directly or indirectly, a target molecule (in the present case PDE or HDAC), by inhibiting catalytic activity. The inhibition of activity can be total if the activity measure when inhibitor compound concentration is up to 10 μM is equal to or below than 10% compared to basal values. If the activity measured is higher than 10% and lower than 100%, more particularly higher than 10% and equal or lower than 90%, the activity is considered partially inhibited.

The term “selective inhibitor compound” as used herein refers to a compound that is able to inhibit a particular isoform of an enzyme target family over others from the same enzyme target family with at least 1 log unit difference in inhibitory potency (IC50).

The term “Pan-HDAC inhibitor compound” as used herein refers to a compound capable of inhibiting totally or partially, at least, one class I HDAC and one class II HDAC.

The term “phosphodiesterases (PDE)” or “cyclic nucleotide phosphodiesterases” as used herein refers to and comprises a group of enzymes that degrade the phosphodiester bond in the second messenger molecules cAMP and cGMP (EC Number 3.1.4.17). They regulate the localization, duration, and amplitude of cyclic nucleotide signaling within subcellular domains. PDEs are therefore important regulators of signal transduction mediated by these second messenger molecules. The superfamily of PDE enzymes is classified into 11 families, namely PDE1-PDE11, in mammals. The classification is based on amino acid sequences, substrate specificities, regulatory properties, pharmacological properties, tissue distribution.

Table 1 summarizes the common names, synonyms, chemical structures, chemical names and CAS Registry numbers for some PDE inhibitor compounds according to the invention.

TABLE 1
PDE inhibitor compounds

| Name, | | | | |
|---|---|---|---|---|
| synonyms & | | | | |
| CAS registry | | | | PDE |
| number | Chemical formula | | Chemical name | Isoform |
| Bay 60-7550 (439083-90-6) | 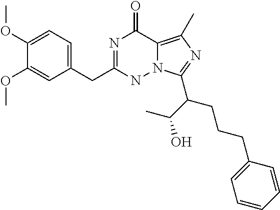 [SMILES: COc1ccc(Cc2nc(=O)c3c(C)nc(C(CCCc4ccccc4)[C@@H](C)O)n3[nH]2)cc1OC] | (1-01) | 2-[(3,4- dimethoxyphenyl)methyl]- 7-[(1R)-1-[(1R)-1- hydroxyethyl]-4-phenyl- butyl]-5-methyl-1H-imidazo [5,1-f][1,2,4]triazin-4-one | PDE2 |
| PF-999 (1394033-54-5) | 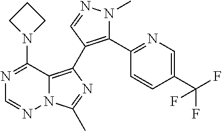 [SMILES: Cc1nc(-c2cnn(C)c2-c2ccc(C(F)(F)F)cn2)c2c(N3CCC3)ncnn12] | (1-02) | 4-(azetidin-1-yl)-7- methyl-5-[1-methyl-5-[5- (trifluoromethyl)-2- pyridyl]pyrazol-4- yl]imidazo[5,1- f][1,2,4]triazine | PDE2 |
| ND-7001 (855170-53-5) | 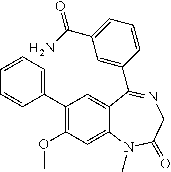 [SMILES: COc1cc2c(cc1-c1ccccc1)C(c1cccc(C(N)=O)c1)=NCC(=O)N2C] | (1-03) | 3-(8-methoxy-1-methyl-2- oxo-7-phenyl-3H-1,4- benzodiazepin-5- yl)benzamide | PDE2 |
| Cilostazol (73963-72-1) | 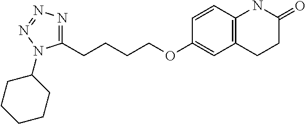 [SMILES: O=C1CCc2cc(OCCCCc3nnnn3C3CCCCC3)ccc2N1] | (1-04) | 6-[4-(1- cyclohexyltetrazol-5- yl)butoxy]-3,4-dihydro- 1H-quinolin-2-one | PDE3 |
| Milrinone (78415-72-2) | 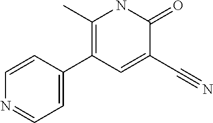 [SMILES: Cc1[nH]c(=O)c(C#N)cc1-c1ccncc1] | (1-05) | 6-methyl-2-oxo-5-(4- pyridyl)-1H-pyridine-3- carbonitrile | PDE3 |
| Enoximone (77671-31-9) | 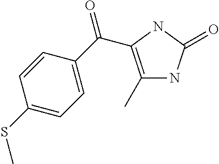 [SMILES: CSc1ccc(C(=O)c2[nH]c(=O)[nH]c2C)cc1] | (1-06) | 4-methyl-5-(4- methylsulfanylbenzoyl)- 1,3-dihydroimidazol-2- one | PDE3 |
| Amrinone Inamrinone (60719-84-8) | 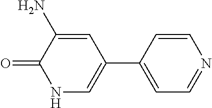 [SMILES: Nc1cc(-c2ccncc2)c[nH]c1=O] | (1-07) | 3-amino-5-(4-pyridyl)-1H- pyridin-2-one | PDE3 |
| Loprinone Olprinone (106730-54-5) | 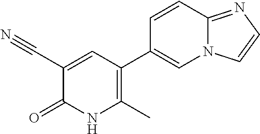 [SMILES: Cc1[nH]c(=O)c(C#N)cc1-c1ccc2nccn2c1] | (1-08) | 5-imidazo[1,2-a]pyridin- 6-yl-6-methyl-2-oxo-1H- pyridine-3-carbonitrile | PDE3 |
| K-134 OPC 33509 (189362-06-9) | 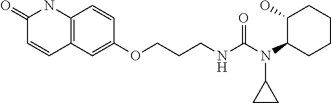 [SMILES: O=C(NCCCOc1ccc2[nH]c(=O)ccc2c1)N(C1CC1)[C@@H]1CCCC[C@H]1O] | (1-09) | 1-cyclopropyl-1-[(1R,2R)- 2-hydroxycyclohexyl]-3- [3-[(2-oxo-1H-quinolin-6- yl)oxy]propyl]urea | PDE3 |
| Rolipram (61413-54-5) | 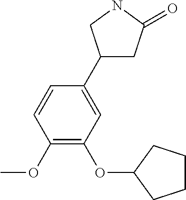 [SMILES: COc1ccc(C2CNC(=O)C2)cc1OC1CCCC1] | (1-10) | 4-[3-(cyclopentoxy)-4- methoxy- phenyl]pyrrolidin-2-one | PDE4 |
| GEBR-7b (1349848-90-3) | 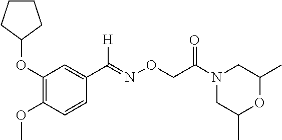 [SMILES: COc1ccc(/C=N/OCC(=O)N2CC(C)OC(C)C2)cc1OC1CCCC1] | (1-11) | 2-[(E)-[3-(cyclopentoxy)- 4-methoxy- phenyl]methyleneamino] oxy-1-(2,6- dimethylmorpholin-4- yl)ethanone | PDE4 |
| GSK356278 (720704-34-7) | 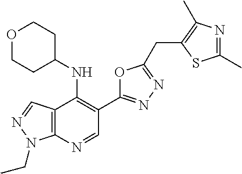 [SMILES: CCn1ncc2c(NC3CCOCC3)c(-c3nnc(Cc4sc(C)nc4C)o3)cnc21] | (1-12) | 5-[5-[(2,4- dimethylthiazol-5- yl)methyl]-1,3,4- oxadiazol-2-yl]-1-ethyl-N- tetrahydropyran-4-yl- pyrazolo[3,4-b]pyridin-4- amine | PDE4 |
| GSK256066 (801312-28-7) | 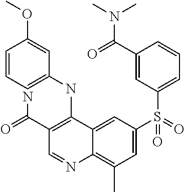 [SMILES: COc1cccc(Nc2c(C(N)=O)cnc3c(C)cc(S(=O)(=O)c4cccc(C(=O)N(C)C)c4)cc23)c1] | (1-13) | 6-[3- (dimethylcarbamoyl) phenyl]sulfonyl-4-(3- methoxyanilino)-8- methyl-quinoline-3- carboxamide | PDE4 |
| Apremilast (608141-41-9) | 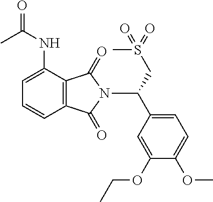 [SMILES: CCOc1cc([C@@H](CS(C)(=O)=O)N2C(=O)c3cccc(NC(C)=O)c3C2=O)ccc1OC] | (1-14) | N-[2-[(1S)-1-(3-ethoxy-4- methoxy-phenyl)-2- methylsulfonyl-ethyl]-1,3- dioxo-isoindolin-4-yl]acetamide | PDE4 |
| MK-0952 (934995-87-6) | 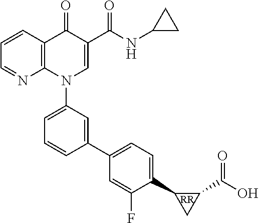 | (1-15) | (1R,2R)-2-[4-[3-[3- (cyclopropylcarbamoyl)- 4-oxo-1,8-naphthyridin-1- yl]phenyl]-2-fluoro-phenyl] cyclopropanecarboxylic acid | PDE4 |
| Roflumilast (162401-32-3) | 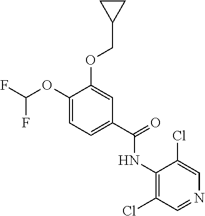 [SMILES: O=C(Nc1c(Cl)cncc1Cl)c1ccc(OC(F)F)c(OCC2CC2)c1] | (1-16) | 3-(cyclopropylmethoxy)- N-(3,5-dichloro-4- pyridyl)-4- (difluoromethoxy)benzamide | PDE4 |
| AN2898 (906673-33-4) | 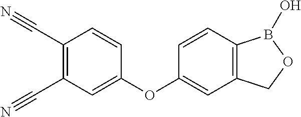 [SMILES: N#Cc1ccc(Oc2ccc3c(c2)COB3O)cc1C#N] | (1-17) | 4-[(1-hydroxy-3H-2,1- benzoxaborol-5- yl)oxy]phthalonitrile | PDE4 |
| AN2728 (906673-24-3) | 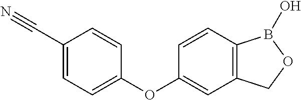 [SMILES: N#Cc1ccc(Oc2ccc3c(c2)COB3O)cc1] | (1-18) | 4-[(1-hydroxy-3H-2,1- benzoxaborol-5- yl)oxy]benzonitrile | PDE4 |
| Ariflo Cilomilast SB207499 (153259-65-5) | 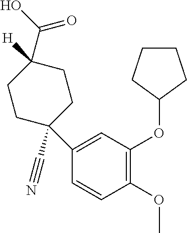 [SMILES: COc1ccc([C@]2(C#N)CC[C@@H](C(=O)O)CC2)cc1OC1CCCC1] | (1-19) | 4-cyano-4-[3- (cyclopentoxy)-4- methoxy-phenyl] cyclohexanecarboxylic acid | PDE4 |
| Dotraverine (14009-24-6) | 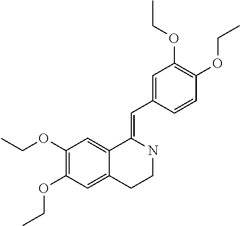 [SMILES: CCOc1ccc(/C=C2\NCCc3cc(OCC)c(OCC)cc32)cc1OCC] | (1-20) | (1Z)-1-[(3,4- diethoxyphenyl)methylene]- 6,7-diethoxy-3,4- dihydro-2H-isoquinoline | PDE4 |
| Ronomilast Elbimilast ELB353 (418794-42-0) | 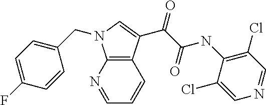 [SMILES: O=C(Nc1c(Cl)cncc1Cl)C(=O)c1cn(Cc2ccc(F)cc2)c2ncccc12] | (1-21) | N-(3,5-dichloro-4- pyridyl)-2-[1-[(4- fluorophenyl)methyl]pyrrolo [2,3-b]pyridin-3-yl]-2- oxo-acetamide | PDE4 |
| Revamilast GRC 4039 (893555-90-3) | 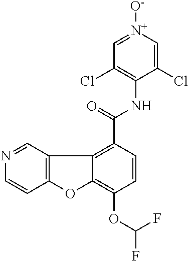 [SMILES: O=C(Nc1c(Cl)c[n+]([O-])cc1Cl)c1ccc(OC(F)F)c2oc3ccncc3c12] | (1-22) | N-(3,5-dichloro-1-oxido- pyridin-1-ium-4-yl)-6- (difluoromethoxy)benzofuro [3,2-c]pyridine-9- carboxamide | PDE4 |
| Tetomilast OPC6535 (145739-56-6) | 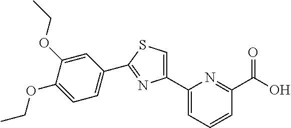 [SMILES: CCOc1ccc(-c2nc(-c3cccc(C(=O)O)n3)cs2)cc1OCC] | (1-23) | 6-[2-(3,4- diethoxyphenyl)thiazol-4- yl]pyridine-2-carboxylic acid | PDE4 |
| E6005 (947620-48-6) | 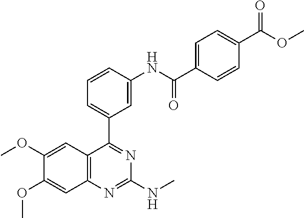 | (1-24) | methyl 4-[[3-[6,7- dimethoxy-2- (methylamino)quinazolin-4- yl]phenyl]carbamoyl]benzoate | PDE4 |
| RPL554 (298680-25-8) | 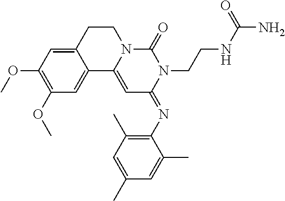 [SMILES: COc1cc2c(cc1OC)-c1c/c(=N\c3c(C)cc(C)cc3C)n(CCNC(N)=O)c(=O)n1CC2] | (1-25) | 2-[(2E)-9,10-dimethoxy- 4-oxo-2-(2,4,6- trimethylphenyl)imino- 6,7-dihydropyrimido[6,1- a]isoquinolin-3- yl]ethylurea | PDE3 and PDE4 |
| GDP-1116 (690690-72-3) | 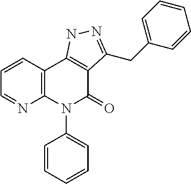 [SMILES: O=c1c2c(Cc3ccccc3)n[nH]c2c2cccnc2n1-c1ccccc1] | (1-26) | 3-benzyl-5-phenyl-1H- pyrazolo[4,3- c][1,8]naphthyridin-4-one | PDE4 |
| HT-0712 IPL455903 (617720-02-2) | 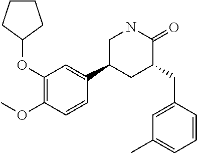 [SMILES: COc1ccc([C@H]2CNC(=O)[C@H](Cc3cccc(C)c3)C2)cc1OC1CCCC1] | (1-27) | (3S,5S)-5-[3- (cyclopentoxy)-4- methoxy-phenyl]-3-(m- tolylmethyl)piperidin-2- one | PDE4 |
| Sildenafil (139755-83-2) | 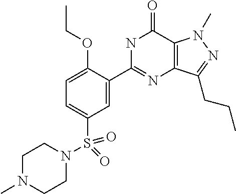 [SMILES: CCCc1nn(C)c2c(=O)[nH]c(-c3cc(S(=O)(=O)N4CCN(C)CC4)ccc3OCC)nc12] | (1-28) | 5-[2-ethoxy-5-(4- methylpiperazin-1- yl)sulfonyl-phenyl]-1- methyl-3-propyl-6H- pyrazolo[4,3-d]pyrimidin- 7-one | PDE5 |
| Vardenafil (224785-90-4) | 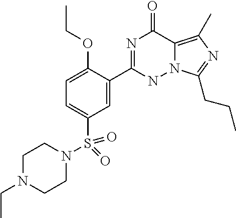 [SMILES: CCCc1nc(C)c2c(=O)nc(-c3cc(S(=O)(=O)N4CCN(CC)CC4)ccc3OCC)[nH]n12] | (1-29) | 2-[2-ethoxy-5-(4- ethylpiperazin-1- yl)sulfonyl-phenyl]-5- methyl-7-propyl-1H- imidazo[5,1- f][1,2,4]triazin-4-one | PDE5 |
| Tadalafil (171596-29-5) | 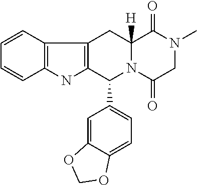 [SMILES: CN1CC(=O)N2[C@H](c3ccc4c(c3)OCO4)c3[nH]c4ccccc4c3C[C@@H]2C1=O] | (1-30) | (6R,12aR)-6-(1,3- Benzodioxol-5-yl)-2- methyl-2,3,6,7,12,12a- hexahydropyrazino[1′,2′: 1,6]pyrido[3,4-b]indole- 1,4-dione | PDE5 |
| Udenafil (268203-93-6) | 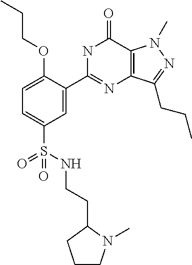 [SMILES: CCCOc1ccc(S(=O)(=O)NCCC2CCCN2C)cc1-c1nc2c(CCC)nn(C)c2c(=O)[nH]1] | (1-31) | 3-(1-methyl-7-oxo-3- propyl-6H-pyrazolo[4,3- d]pyrimidin-5-yl)-N-[2-(1- methylpyrrolidin-2- yl)ethyl]-4-propoxy- benzenesulfonamide | PDE5 |
| Avanafil (330784-47-9) | 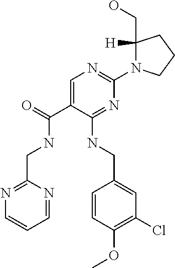 [SMILES: COc1ccc(CNc2nc(N3CCC[C@H]3CO)ncc2C(=O)NCc2ncccn2)cc1Cl] | (1-32) | 4-[(3-chloro-4-methoxy- phenyl)methylamino]-2- [(2S)-2- (hydroxymethyl)pyrrolidin- 1-yl]-N-(pyrimidin-2- ylmethyl)pyrimidine-5- carboxamide | PDE5 |
| Mirodenafil (862189-95-5) | 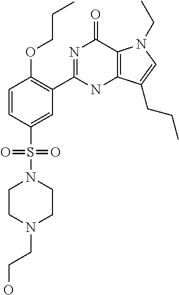 [SMILES: CCCOc1ccc(S(=O)(=O)N2CCN(CCO)CC2)cc1-c1nc(=O)c2c([nH]1)c(CCC)cn2CC] | (1-33) | 5-ethyl-2-[5-[4-(2- hydroxyethyl)piperazin-1- yl]sulfonyl-2-propoxy- phenyl]-7-propyl-1H- pyrrolo[3,2-d]pyrimidin-4- one | PDE5 |
| Lodenafil (139755-85-4) | 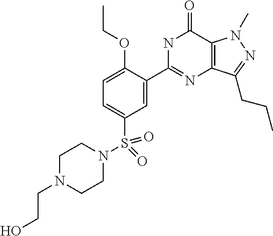 [SMILES: CCCc1nn(C)c2c(=O)[nH]c(-c3cc(S(=O)(=O)N4CCN(CCO)CC4)ccc3OCC)nc12] | (1-34) | 5-[2-ethoxy-5-[4-(2- hydroxyethyl)piperazin-1- yl]sulfonyl-phenyl]-1- methyl-3-propyl-6H- pyrazolo[4,3-d]pyrimidin- 7-one | PDE5 |
| Dasantafil Sch 446132 (569351-91-3) | 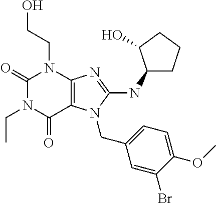 [SMILES: CCn1c(=O)c2c(nc(N[C@@H]3CCC[C@H]3O)n2Cc2ccc(OC)c(Br)c2)n(CCO)c1=O] | (1-35) | 7-[(3-bromo-4-methoxy- phenyl)methyl]-1-ethyl-8- [[(1R,2R)-2- hydroxycyclopentyl]amino]- 3-(2- hydroxyethyl)purine-2,6- dione | PDE5 |
| PF-00489791 (1198359-14-6) | 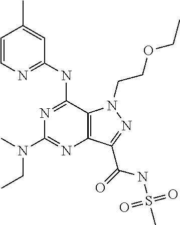 [SMILES: CCOCCn1nc(C(=O)NS(C)(=O)=O)c2nc(N(C)CC)nc(Nc3cc(C)ccn3)c21] | (1-36) | 1-(2-ethoxyethyl)-5- [ethyl(methyl)amino]-7- [(4-methyl-2- pyridyl)amino]-N- methylsulfonyl- pyrazolo[4,3- d]pyrimidine-3- carboxamide | PDE5 |
| S-14 (18741-24-7) | 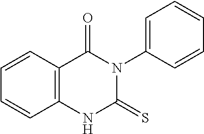 [SMILES: O=c1c2ccccc2[nH]c(=S)n1-c1ccccc1] | (1-37) | 3-phenyl-2-thioxo-1H- quinazolin-4-one | PDE7 |
| PF-04957325 (1305115-80-3) | 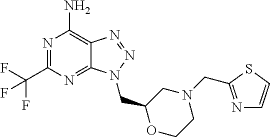 [SMILES: Nc1nc(C(F)(F)F)nc2c1nnn2C[C@H]1CN(Cc2nccs2)CCO1] | (1-38) | 3-[[(2R)-4-(thiazol-2- ylmethyl)morpholin-2- yl]methyl]-5- (trifluoromethyl)triazolo[4, 5-d]pyrimidin-7-amine | PDE8 |
| PF-04447943 (1082744-20-4) | 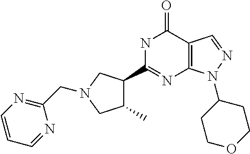 [SMILES: C[C@@H]1CN(Cc2ncccn2)C[C@H]1c1nc2c(cnn2C2CCOCC2)c(=O)[nH]1] | (1-39) | 6-[(3S,4S)-4-methyl-1- (pyrimidin-2- ylmethyl)pyrrolidin-3-yl]- 1-tetrahydropyran-4-yl- 5H-pyrazolo[3,4- d]pyrimidin-4-one | PDE9 |
| PF-02545920 (898562-94-2) | 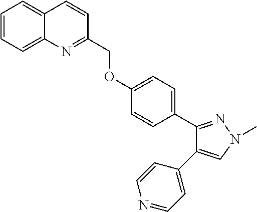 [SMILES: Cn1cc(-c2ccncc2)c(-c2ccc(OCc3ccc4ccccc4n3)cc2)n1] | (1-40) | 2-[[4-[1-methyl-4-(4- pyridyl)pyrazol-3- yl]phenoxy]methyl]quinoline | PDE10 |
| RG7203 | (1-41) | | | PDE10 |
| TAK-063 | (1-42) | | | PDE10 |
| OMS824 | (1-43) | | | PDE10 |
| FRM-6308 | (1-44) | | | PDE10 |
| BI409306 | (1-45) | | | PDE9 |
| MK-0873 (500355-52-2) | 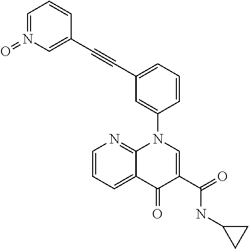 [SMILES: O=C(NC1CC1)c1cn(-c2cccc(C#Cc3ccc[n+]([O-])c3)c2)c2ncccc2c1=O] | (1-46) | N-cyclopropyl-4-oxo-1-[3- [2-(1-oxo-3- pyridyl)ethynyl]phenyl]- 1,8-naphthyridine-3- carboxamide | PDE4 |
| AMG579 (1227067-61-9) | 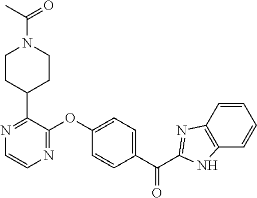 [SMILES: CC(=O)N1CCC(c2nccnc2Oc2ccc(C(=O)c3nc4ccccc4[nH]3)cc2)CC1] | (1-47) | 1-[4-[3-[4-(1H- benzimidazole-2- carbonyl)phenoxy]pyrazin- 2-yl]-1- piperidyl]ethanone | PDE10 |

The compounds of formula (1-01), (1-04)-(1-08), (1-10)-(1-23), (1-28)-(1-35), (1-37)-(1-40) are commercially available. The compounds of formula (1-02), (1-03), (1-09), (1-24), (1-25), (1-26), (1-27), (1-36), (1-46) and (1-47) can be synthetized by methods well-known in the art. For example, the compound of formula (1-02) may be synthetized as described in US 20120214791. For example, the compound of formula (1-03) may be synthetized as described in EP 1548011. For example, the compound of formula (1-09) may be synthetized as described in WO 9712869. For example, the compound of formula (1-24) may be synthetized as described in WO 2007097317. For example, the compound of formula (1-25) may be synthetized as described in WO 2000058308. For example, the compound of formula (1-26) may be synthetized as described in WO 2004041819. For example, the compound of formula (1-27) may be synthetized as described in US 20030186943. For example, the compound of formula (1-36) may be synthetized as described in WO 2005049616. For example, the compound of formula (1-46) may be synthetized as described in WO 2003018579. For example, the compound of formula (1-47) may be synthetized as described in WO 2010057121.

The ability of the compounds of formula (1-01) to (1-47) of inhibiting PDE activity is disclosed for example in the bibliographic references summarized in the table below.

TABLE 2
Bibliographic references of inhibiting activity of the compounds

| Compound of | |
|---|---|
| formula | Bibliographic reference |
| (1-01), (1-04), (1- | Maurice, DH et al. Nat Rev Drug Discov. 2014, vol. 13, pp. 290-314. |
| 05), (1-06), (1- | |
| 10), (1-12), (1- | |
| 13), (1-14), (1- | |
| 15), (1-16), (1- | |
| 17), (1-18), (1- | |
| 19), (1-23), (1- | |
| 25), (1-28), (1- | |
| 29), (1-30), (1- | |
| 31), (1-32), (1- | |
| 33), (1-36), (1- | |
| 38), (1-39), (1- | |
| 40) | |
| (1-02), (1-03) | Gomez, L et al. Bioorg Med Chem Lett. 2013, vol. 23, pp. 6522-7 |
| (1-07) | Thompson, PE et al. Curr Top Med Chem. 2007, vol. 7, pp. 421-36 |
| (1-08) | Endoh, M. Cardiovascular Drug Reviews 1993, vol. 11, pp. 432-50 |
| (1-09) | Sasaki, Y. European Journal of Pharmacology 2012, vol. 689, pp. |
| | 132-138 |
| (1-11) | Bruno, O et al. J Med Chem. 2009, vol. 52, pp. 6546-57. |
| (1-20) | Kapui, Z et al. Neurobiology 1999, vol. 7, pp. 71-73. |
| (1-21) | WO2002034747 |
| (1-22) | WO2006064355 |
| (1-24) | Ishii, NJ et al. Pharmacol Exp Ther. 2013, vol. 346, pp. 105-12. |
| (1-26) | WO2004041819 |
| (1-27) | Giembycz, MA. British Journal of Pharmacology 2008, vol 155, pp. |
| | 288-290 |
| (1-34) | Toque, HA. Eur J Pharmacol. 2008, vol. 591, pp. 189-195 |
| (1-35) | WO2003101991 |
| (1-37) | WO2010133742 |
| (1-41) | http://www.biocentury.com/products/rg7203 |
| (1-42) | http://www.biocentury.com/products/tak-063 |
| (1-43) | http://www.biocentury.com/products/oms824 |
| (1-44) | http://www.biocentury.com/products/evp-6308 |
| (1-45) | https://www.bioehringer- |
| | ingelheim.com/research_development/drug_discovery/pipeline.html |
| (1-46) | Guay, D. et al. Bioorg Med Chem Lett. 2008, vol. 18, pp. 5554-8 |
| (1-47) | Hu, E. et al. J Med Chem. 2014, vol. 57, pp. 6632-41 |

In one embodiment, optionally in combination with any of the embodiments above or below, the PDE inhibitor compound is selected from the group consisting of compound (1-01) to (1-44).

In one embodiment, optionally in combination with any of the embodiments above or below, the PDE inhibitor compound is selected from the group consisting of compound (1-01) to (1-40), (1-46) and (1-47).

In one embodiment, optionally in combination with any of the embodiments above or below, the PDE inhibitor compound is selective for a PDE selected from the group consisting of PDE1, PDE2, PDE3, PDE4, PDE5, PDE6, PDE7, PDE8, PDE9, PDE10, and PDE11. In a preferred embodiment the PDE inhibitor compound is a selective PDE5 inhibitor compound (formulas (1-28) to (1-36)), more preferably Tadalafil (formula (1-30)), Sildenafil (formula (1-28)) or Vardenafil (formula (1-29)).

The term “Histone deacetylase (HDAC)” as used herein refers to and comprises a group of enzymes that remove acetyl groups (0=C—CH₃) from a ε-N-acetyl lysine amino acid on a histone, allowing the histones to wrap the DNA more tightly (EC Number 3.5.1.98). This is important because DNA is wrapped around histones, and DNA expression is regulated by acetylation and de-acetylation.

HDACs are classified in four classes I to IV based on function and DNA sequence similarity. Class I includes isoforms HDAC1, HDAC2, HDAC3, and HDAC8; class II includes HDAC4, HDAC5, HDAC6, HDAC7, HDAC9 and HDAC10; Class IV includes HDAC11.

Table 3 summarizes the common names, synonyms, chemical structures, chemical names and CAS Registry numbers for some HDAC inhibitor compounds according to the invention.

TABLE 3
HDAC inhibitor compounds

| Name, | | | | |
|---|---|---|---|---|
| synonyms & | | | | |
| CAS registry | | | Chemical | HDAC |
| number | Chemical formula | | name | Isoform |
| Vorinostat SAHA (149647-78-9) | 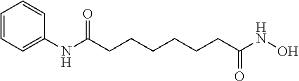 [SMILES: O=C(CCCCCCC(=O)Nc1ccccc1)NO] | (2-01) | 8- (hydroxyamino)- 8-oxo-N- phenyl- octanamide | pan-HDAC (HDAC1, HDAC2 HDAC3, and HDAC6) |
| Romidepsin (128517-07-7) | 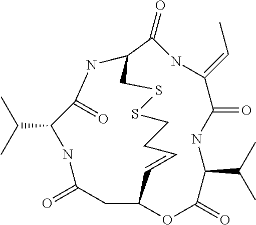 [SMILES: C/C=C1\NC(=O)[C@H]2CSSCC/C=C/[C@H](CC(=O)N[C@H](C(C)C)C(=O)N2)OC(=O)[C@H](C(C)C)NC1=O] | (2-02) | (1S,4S,7Z,10S, 16E,21R)-7- ethylidene- 4,21- diisopropyl-2- oxa-12,13- dithia- 5,8,20,23- tetrazabicyclo [8.7.6]tricos- 16-ene- 3,6,9,19,22- pentone | Class-I (HDAC1, HDAC2, HDAC3 and HDAC8) |
| Panobinostat (404950-80-7) | 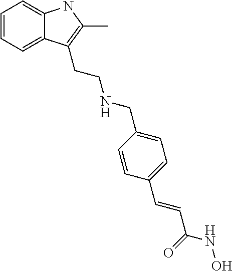 [SMILES: Cc1[nH]c2ccccc2c1CCNCc1ccc(/C=C/C(=O)NO)cc1] | (2-03) | (E)-3-[4-[[2-(2- methyl-1H- indol-3- yl)ethylamino] methyl]phenyl] prop-2- enehydroxamic acid | pan-HDAC (HDAC1, HDAC2, HDAC3, and HDAC6) |
| Entinostat MS-275 (209783-80-2) | 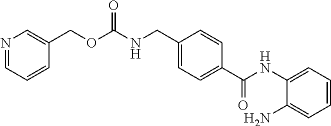 [SMILES: Nc1ccccc1NC(=O)c1ccc(CNC(=O)OCc2cccnc2)cc1] | (2-04) | 3- pyridylmethyl N-[[4-[(2- aminophenyl) carbamoyl] phenyl]methyl] carbamate | Class-I (HDAC1 and HDAC2) |
| Mocetinostat (726169-73-9) | 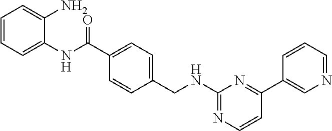 [SMILES: Nc1ccccc1NC(=O)c1ccc(CNc2nccc(-c3cccnc3)n2)cc1] | (2-05) | N-(2- aminophenyl)- 4-[[[4-(3- pyridyl) pyrimidin-2-yl] amino]methyl] benzamide | Class-I (HDAC1 and HDAC2) |
| Rocilinostat ACY-1215 (1316214-52-4) | 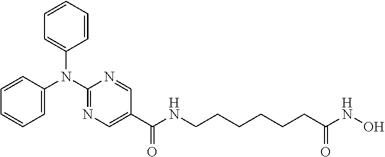 [SMILES: O=C(CCCCCCNC(=O)c1cnc(N(c2ccccc2)c2ccccc2)nc1)NO] | (2-06) | N-[7- (hydroxyamino)- 7-oxo- heptyl]-2-(N- phenylanilino) pyrimidine-5- carboxamide | HDAC6 |
| Belinostat (866323-14-0) | 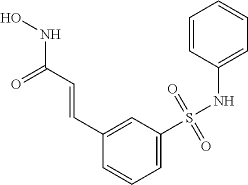 [SMILES: O=C(/C=C/c1cccc(S(=O)(=O)Nc2ccccc2)c1)NO] | (2-07) | (E)-3-[3- (phenylsulfamoyl) phenyl]prop-2- enehydroxamic acid | pan-HDAC (HDAC1, HDAC2, HDAC3 and HDAC6) |
| Resminostat (864814-88-0) | 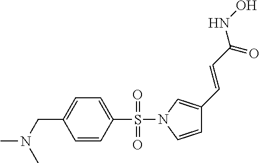 [SMILES: CN(C)Cc1ccc(S(=O)(=O)n2ccc(/C=C/C(=O)NO)c2)cc1] | (2-08) | (E)-3-[1-[4- [(dimethylamino) methyl]phenyl] sulfonylpyrrol- 3-yl]prop-2- enehydroxamic acid | pan-HDAC (HDAC1, HDAC3 and HDAC6) |
| Givinostat ITF2357 (497833-27-9) | 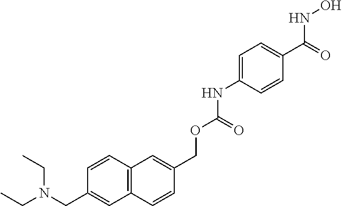 [SMILES: CCN(CC)Cc1ccc2cc(COC(=O)Nc3ccc(C(=O)NO)cc3)ccc2c1] | (2-09) | [6- (diethylamino methyl)-2- naphthyl]methyl N-[4- (hydroxycarbamoyl) phenyl]carbamate | pan-HDAC |
| Pracinostat SB939 (929016-96-6) | 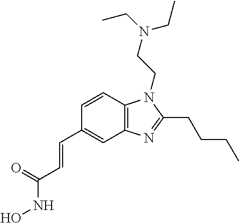 [SMILES: CCCCc1nc2cc(/C=C/C(=O)NO)ccc2n1CCN(CC)CC] | (2-10) | (E)-3-[2-butyl- 1-[2- (diethylamino) ethyl]benzimidazol- 5-yl]prop- 2- enehydroxamic acid | pan-HDAC (HDAC1, HDAC2, HDAC3, HDAC8, HDAC4, HDAC5, HDAC6, HDAC7, HDAC9, HDAC10, HDAC11) |
| Abexinostat PCI-24781 (783355-60-2) | 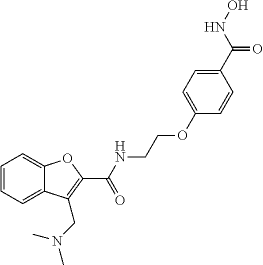 [SMILES: CN(C)Cc1c(C(=O)NCCOc2ccc(C(=O)NO)cc2)oc2ccccc12] | (2-11) | 3- [(dimethylamino) methyl]-N- [2-[4- (hydroxycarbamoyl) phenoxy] ethyl]benzofuran- 2- carboxamide | pan-HDAC (HDAC1, HDAC2, HDAC3, HDAC6, HDAC8, and HDAC10) |
| 4SC-202 (910462-43-0) | 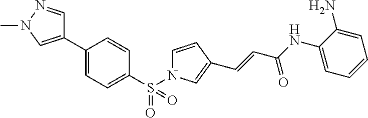 [SMILES: Cn1cc(-c2ccc(S(=O)(=O)n3ccc(/C=C/C(=O)Nc4ccccc4N)c3)cc2)cn1] | (2-12) | (E)-N-(2- aminophenyl)- 3-[1-[4-(1- methylpyrazol- 4- yl)phenyl] sulfonylpyrrol-3- yl]prop-2- enamide | Class-I (HDAC1, HDAC2, and HDAC3) |
| AR-42 (935881-37-1) | 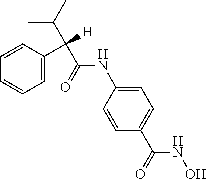 [SMILES: CC(C)[C@H](C(=O)Nc1ccc(C(=O)NO)cc1)c1ccccc1] | (2-13) | (2S)-N-[4- (hydroxycarbamoyl) phenyl]- 3-methyl-2- phenyl- butanamide | pan-HDAC |
| CG200745 (936221-33-9) | 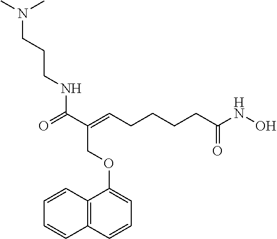 [SMILES: CN(C)CCCNC(=O)/C(=C/CCCCC(=O)NO)COc1cccc2ccccc12] | (2-14) | (E)-N-[3- (dimethylamino) propyl]-8- (hydroxyamino)- 2-(1- naphthyloxymethyl)- 8-oxo- oct-2-enamide | pan-HDAC |
| Tubastatin A (1252003-15-8) | 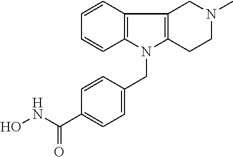 [SMILES: CN1CCc2c(c3ccccc3n2Cc2ccc(C(=O)NO)cc2)C1] | (2-15) | 4-[(2-methyl- 3,4-dihydro- 1H-pyrido[4,3- b]indol-5- yl)methyl] benzenecarbohydroxamic acid | HDAC6 |
| Sodium phenylbutyrate (1716-12-7) | 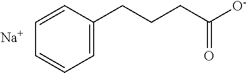 [SMILES: [Na+] ; O=C([O-])CCCc1ccccc1] | (2-16) | sodium; 4- phenylbutanoate | pan-HDAC |
| Valproic acid VP101 (99-66-1) | 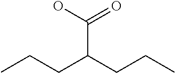 | (2-17) | 2- propylpentanoic acid | pan-HDAC |
| Chidamide CS055 HBI-8000 (743420-02-2) | 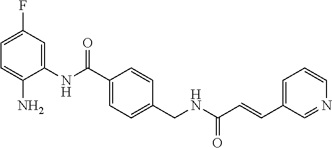 [SMILES: Nc1ccc(F)cc1NC(=O)c1ccc(CNC(=O)/C=C/c2cccnc2)cc1] | (2-18) | N-(2-amino-5- fluoro-phenyl)- 4-[[[(E)-3-(3- pyridyl)prop-2- enoyl]amino] methyl]benzamide | pan-HDAC (HDAC1, HDAC2, HDAC3, and HDAC10) |
| Quisinostat JNJ-26481585 (875320-29-9) | 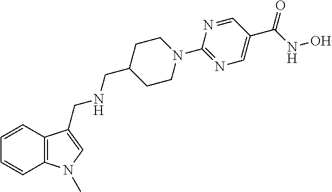 [SMILES: Cn1cc(CNCC2CCN(c3ncc(C(=O)NO)cn3)CC2)c2ccccc21] | (2-19) | 2-[4-[[(1- methylindol-3- yl)methylamino] methyl]-1- piperidyl]pyrimidine- 5-carbohydroxamic acid | pan-HDAC |
| Tefinostat CHR-2845 (914382-60-8) | 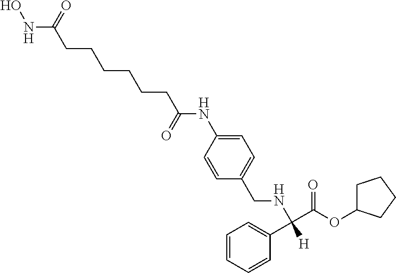 [SMILES: O=C(CCCCCCC(=O)Nc1ccc(CN[C@H](C(=O)OC2CCCC2)c2ccccc2)cc1)NO] | (2-20) | cyclopentyl (2S)-2-[[4-[[8- (hydroxyamino)- 8-oxo- octanoyl]amino] phenyl] methylamino]-2- phenyl-acetate | pan-HDAC |
| CHR-3996 (1235859-13-8) | 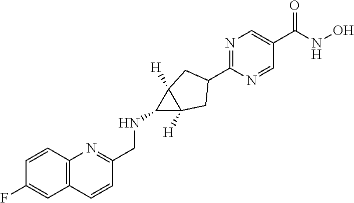 [SMILES: O=C(NO)c1cnc(C2C[C@@H]3[C@H](C2)[C@H]3NCc2ccc3cc(F)ccc3n2)nc1] | (2-21) | 2-[(1R,5S)-6- [(6-fluoro-2- quinolyl) methylamino]-3- bicyclo[3.1.0] hexanyl]pyrimidine-5- carbohydroxamic acid | Class-I |
| RG2833 RGFP 109 (1215493-56-3) | 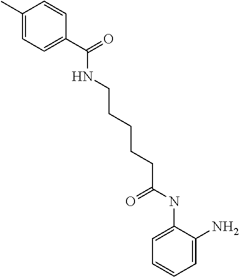 [SMILES: Cc1ccc(C(=O)NCCCCCC(=O)Nc2ccccc2N)cc1] | (2-22) | N-[6-(2- aminoanilino)- 6-oxo-hexyl]- 4-methyl- benzamide | Class-I (HDAC1 and HDAC3) |
| Tacedinaline CI-994 112522-64-2 | 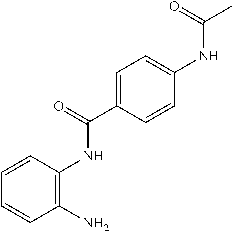 [SMILES: CC(=O)Nc1ccc(C(=O)Nc2ccccc2N)cc1] | (2-23) | 4-acetamido- N-(2- aminophenyl) benzamide | Class-I (HDAC1 and HDAC2) |

| | | |
|---|---|---|
| ACY-241 | (2-24) | HDAC6 |
| OCID-4681 | (2-25) | Class-1 |
| | | HDAC1 |
| | | and |
| | | HDAC2) |
| FRM-0334 | (2-26) | HDAC |
| EVP-0334 | | (class I) |
| 1678555-75-3 | | |

The compounds of formula (2-01)-(2-11), (2-13)-(2-19) and (2-21)-(2-23) are commercially available. The compounds of formula (2-12) and (2-20) can be synthetized by methods well-known in the art. For example, the compound of formula (2-12) may be synthetized as described in WO2006097474. For example, the compound of formula (2-20) may be synthetized as described in WO2006117567.

The ability of the compounds of formula (2-01) to (2-26) of inhibiting HDAC activity is disclosed for example in the bibliographic references summarized in the table below.

TABLE 4
Bibliographic references of inhibiting activity of the compounds

| Compound of | |
|---|---|
| formula | Bibliographic reference |
| (2-01)-(2-14), | Arrowsmith CH et al. Nat Rev Drug Discov. 2012, |
| (2-17), (2-22) | vol. 11, pp. 384-400 |
| (2-15) | Vishwakarma S et al. Int Immunopharmacol. 2013, vol. |
| | 16, pp. 72-8. |
| (2-16) | Gore SD et al. Expert Opin Investig Drugs. 2000, vol. 9, |
| | pp. 2923-34. |
| (2-18) | WO2004071400 |
| (2-19) | Arts Jet al. Clin Cancer Res. 2009, pp. 15, vol. 6841-51 |
| (2-20) | Ossenkoppele GJ et al. Br J Haematol. 2013, vol. 162, |
| | pp. 191-201 |
| (2-21) | Banerji, U et al. Clinical Cancer Research 2012, vol. 18, |
| | pp. 2687-2694 |
| (2-23) | Kraker AJ et al. Molecular Cancer Therapeutics. 2003, |
| | vol. 2(4), pp. 401-408 |
| (2-24) | http://adisinsight.springer.com/drugs/800042221 |
| (2-25) | Narayanan S. Annals of Oncology. 2010, vol. 21, |
| | suppl. 2, pp. ii34-38 |
| (2-26) | http://www.biocentury.com/products/frm-0334 |

In one embodiment, optionally in combination with any of the embodiments above or below, the HDAC inhibitor compound is selected from the group consisting of compound (2-01) to (2-22).

In one embodiment, optionally in combination with any of the embodiments above or below, the HDAC inhibitor compound is selected from the group consisting of compound (2-01) to (2-23).

In one embodiment, optionally in combination with any of the embodiments above or below, the HDAC inhibitor compound is a Pan-HDAC inhibitor. In a preferred embodiment, this Pan-HDAC inhibitor compound is selected from the group consisting of compounds of Vorinostat (formula (2-01)), Panobinostat (formula (2-03)), Belinostat (formula (2-07)) and Valproic acid (formula (2-17)); more preferably Vorinostat.

In another embodiment, optionally in combination with any of the embodiments above or below, the HDAC inhibitor compound is a class-I inhibitor, and in particular compound Romidepsin (formula (2-02)).

In one preferred embodiment, optionally in combination with any of the embodiments above or below, Vorinostat (formula (2-01)) is combined with a selective PDE5 inhibitor compound, more preferable with Tadalafil (formula (1-30)), Sildenafil (formula (1-28)) or Vardenafil (formula (1-29)).

In another preferred embodiment, optionally in combination with any of the embodiments above or below, Panobinostat (formula (2-03)) is combined with a selective PDE5 inhibitor compound, more preferable with Tadalafil (formula (1-30)), Sildenafil (formula (1-28)) or Vardenafil (formula (1-29)).

According to the invention, the PDE inhibitor compound and HDAC inhibitor compound as defined above can be used as combination or combined preparation for the treatment and/or prevention of neurological disorders coursing with a cognition deficit or impairment, or neurodegenerative diseases, and can be used in a broad range of therapeutic applications.

In a particular embodiment, the neurodegenerative diseases are neurodegenerative diseases coursing with a cognition deficit or impairment. More particularly, the neurodegenerative disease or neurological disorder coursing with a cognition deficit or impairment is selected from Alzheimer's disease, Parkinson's disease, Huntington's disease, vascular dementia (uncomplicated, with delirium, with delusions or with depressed mood), mild cognitive impairment and age-associated cognition impairment. More preferably, the disease is Alzheimer's disease.

In one embodiment of the invention, optionally in combination with any of the embodiments above or below, the PDE inhibitor compound and/or the HDAC inhibitor compound as defined above are active pharmaceutical or veterinary ingredients of a pharmaceutical or veterinary composition, which comprises therapeutically effective amounts of the PDE inhibitor compound, and/or of the HDAC inhibitor compound, together with one or more pharmaceutically or veterinary acceptable excipients or carriers.

The expression “therapeutically effective amount” as used herein, refers to the amounts of a PDE inhibitor compound and/or a HDAC inhibitor compound that, when administered as a combination or combined preparation, are sufficient to prevent development of, or alleviate to some extent, one or more of the symptoms of the disease which is addressed. The specific doses of the PDE inhibitor compound and of the HDAC inhibitor compound of the invention to obtain a therapeutic benefit may vary depending on the particular circumstances of the individual patient including, among others, the size, weight, age and sex of the patient, the nature and stage of the disease, the aggressiveness of the disease, and the route of administration. For example, a dose of from about 0.01 to about 300 mg/kg of PDE inhibitor compound and a dose of from about 0.01 to about 300 mg/kg of HDAC inhibitor compound may be used.

The expression “pharmaceutically or veterinary acceptable excipients or carriers” refers to pharmaceutically or veterinary acceptable materials, compositions or vehicles. Each component must be pharmaceutically or veterinary acceptable in the sense of being compatible with the other ingredients of the pharmaceutical or veterinary composition. It must also be suitable for use in contact with the tissue or organ of humans and animals without excessive toxicity, irritation, allergic response, immunogenicity or other problems or complications commensurate with a reasonable benefit/risk ratio.

The election of the pharmaceutical or veterinary formulation will depend upon the nature of the active compound and its route of administration. Any route of administration may be used. In one embodiment of the invention, optionally in combination with any of the embodiments above or below, the pharmaceutical or veterinary composition is administered orally, topically or parenterally.

For example, the pharmaceutical or veterinary composition may be formulated for oral administration and may contain one or more physiologically compatible carriers or excipients, in solid or liquid form. These preparations may contain conventional ingredients such as binding agents, fillers, lubricants, and acceptable wetting agents.

The pharmaceutical or veterinary composition may be formulated for parenteral administration in combination with conventional injectable liquid carriers, such as water or suitable alcohols. Conventional pharmaceutical or veterinary excipients for injection, such as stabilizing agents, solubilizing agents, and buffers, may be included in such compositions. These pharmaceutical or veterinary compositions may be injected subcutaneously, intramuscularly, intraperitoneally, or intravenously.

The pharmaceutical or veterinary composition may be formulated for topical administration. Formulations include creams, lotions, gels, powders, solutions and patches wherein the compound is dispersed or dissolved in suitable excipients. The topical compositions of the invention may be administered by means of a carrier material, which can be a solid support. Thus, it also forms part of the invention a topical composition comprising a carrier material, which can be a solid support. Illustrative, non-limiting examples of solid supports include intelligent textiles, dressings, coatings, sponges, band-aids, sanitary pads, compresses, plasters, etc. The manufacture of such compositions can be obtained by conventional methods, for example, by mixing the combinations of the invention and the material carrier.

The pharmaceutical or veterinary compositions may be in any form, including, among others, tablets, pellets, capsules, aqueous or oily solutions, suspensions, emulsions, or dry powdered forms suitable for reconstitution with water or other suitable liquid medium before use, for immediate or retarded release.

The appropriate excipients and/or carriers, and their amounts, can readily be determined by those skilled in the art according to the type of formulation being prepared.

Throughout the description and claims the word “comprise” and variations of thereof, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### Examples

### In-Vitro Assay Using Primary Neuronal Cultures and the Cell Line SHSY-5Y HDAC Activity in Wild Type Neurons

The cellular assay to determine HDAC activity was assessed by Western blot analysis using a specific antibody against acetylated histone 3 at Lys9 (AcH3K9, Cell Signalling).

Primary neuronal cultures derived from the hippocampus and cortex of embryonic day 16 of wild type mice were used. Tissue was triturated using glass pipettes until neurons were dissociated. Neurons were plated with serum-free neurobasal media with B27 supplement (Invitrogen, Gaithersburg, Md.) and 2 mM L-glutamine on poly-L-lysine-treated (0.1 mg/ml; Sigma) 35 mm dishes.

To determine the concentration-response curve effect of Vorinostat and Tadalafil, cell cultures of 15 days in vitro were treated at different concentrations (10, 100 and 500 nM) during 2 h (Table 5).

Once concentration of Vorinostat that does not lead to increasing AcH3 level vs basal is identified; this minimal concentration is utilized in combination with Tadalafil to identify synergistic effect in histone 3 acetylation. Thus, to determine if a synergistic effect on epigenetic mark (AcH3) exists when both compounds are combined, Vorinostat at 50 nM was combined with Tadalafil at different concentrations (12.5, 25, 50, 100 and 200 nM) during 2 h (Table 6).

### HDAC Activity in SHSY-5Y

A human neuroblastoma cell line (SHSY-5Y) was also used. Cell line was obtained from ATCC (CRL-2266) (Biedler et al., Cancer Research, 38, 3751-3757 1978) and cultured in 35 mm plates (Becton Dickinson, N.J.). The cells were grown to 90% confluence at 37° C. in an atmosphere of 5% CO₂ and in Dulbecco's modified Eagle's medium supplemented with Glutamax (Gibco, Invitrogen, CA), 100 units/ml penicillin/streptomycin, 1× Minimum Essential Media (MEM) non essential amino acids and 10% fetal bovine serum (Gibco, Invitrogen, CA).

Cells were collected in a buffer containing a cold lysis buffer with protease inhibitors (0.2 M NaCl, 0.1 M HEPES (4-(2-hydroxyethyl)-1-piperazineethanesulfonic acid), 10% glycerol, 200 mM NaF, 2 mM Na₄P2O₇, 5 mM EDTA (ethylenediaminetetraacetic acid), 1 mM EGTA (ethylene glycol tetraacetic acid), 2 mM DTT (dithiothreitol), 0.5 mM PMSF (phenylmethylsulfonyl fluoride), 1 mM Na₃VO₄ and Complete Protease Inhibitor Cocktail, Roche Diagnostics), centrifuged at 14,000×g 4° C. for 20 min and the supernatant was aliquoted and stored at −80° C. Total protein concentrations were determined using the BioRad Bradford protein assay (BioRad Laboratories).

Protein samples were mixed with Laemmli sample buffer, resolved onto SDS-polyacrylamide gels and transferred to nitrocellulose membrane. The membranes were blocked with 5% milk, 0.05% Tween-20 in PBS (Phosphate-Buffered Saline) or TBS (Tris-Buffered Saline) followed by overnight incubation with the following primary antibodies: rabbit polyclonal anti-acetylated Histone 3 (acetyl K9, Cell Signalling 1:1000), mouse monoclonal anti-actin (Sigma, 1:50000) in the corresponding buffer. Following two washes in TBS/Tween20 and one wash in TBS alone, immunolabeled protein bands were detected by using HRP-conjugated anti-rabbit or anti-mouse antibody (Santa Cruz; dilution 1:5000) following an enhanced chemiluminescence system (ECL, GE Healthcare Bioscience, Buckinghamshire, UK), and autoradiographic exposure to HyperfilmtECL (GE Healthcare Bioscience). Quantity One™ software v.4.6.3 (Bio-Rad) was used for quantification.

Once concentration of HDAC inhibitor that does not lead to increasing AcH3 level vs basal is determined to be 10 nM; this minimal concentration is utilized in combination with PDE inhibitors to identify synergistic effect in histone 3 acetylation. Cell line was treated during 2 hours with the combination of an HDAC inhibitor and PDE inhibitor at different concentrations as indicated in Table 7 showing the synergistic effects in histone acetylation.

Table 5 shows increment in Histone 3 acetylation level (AcH3) vs basal (no treatment) using wild-type neurons; thus, basal ×1 means no increment (+), basal ×2≦(++).

TABLE 5
AcH3 increment by PDE and HDAC inhibitors alone using
wild-type neurons

| | AcH3^{a} | AcH3^{b} | AcH3^{c} |
|---|---|---|---|
| Vorinostat | + | ++ | ++ |
| Tadalafil | + | + | + |

^{a}10 nM,
^{b}100 nM and
^{c}500 nM

Table 6 shows increment in Histone 3 acetylation level (AcH3) vs basal (no treatment) using wild-type neurons; thus, basal ×1 means no increment (+), basal ×2≦(++)<basal ×3, basal ×3≦(+++)<basal ×4 and basal ×4≦(++++). N.E. means No Effect.

TABLE 6
AcH3 increment by PDE and HDAC combinations
using wild-type neurons

| | V^{a} | V^{a} + T^{b} | V^{a} + T^{c} | V^{a} + T^{d} | V^{a} + T^{e} | V^{a} + T^{f} |
|---|---|---|---|---|---|---|
| AcH3 | + | + | ++ | ++ | ++ | ++++ |

V, Vorinostat; T, Tadalafil
^{a}50 nM,
^{b}12.5 nM,
^{c}25 nM,
^{d}50 nM,
^{e}100 nM and
^{f}200 nM

Table 7 also shows increment in Histone 3 acetylation level (AcH3) vs basal (no treatment) using SHSY-5Y cell line; thus, basal ×1 means no increment (+), basal ×2≦(++)<basal ×3, basal ×3≦(+++)<basal ×4 and basal ×4≦(++++). N.E. means No Effect.

TABLE 7
AcH3 increment by PDE and HDAC combinations
using SHSY-5Y cell line

| | AcH3 |
|---|---|
| V^{a} | + |
| V^{a} + Vd^{b} | +++ |
| V^{a} + S^{c} | ++ |

V. Vorinostat;
Vd, Vardenafil;
S. Sildenafil
^{a}10 nM,
^{b}10 nM,
^{c}100 nM

As shown in Table 7 there is a clear synergistic effect when both compounds are combined.

### In Vivo Studies Using Tg2576 AD Mouse Model

### Memory Function in Aged-Tg2576 Mice

The effect on memory function of Vorinostat (12.5 mg/kg, i.p.), Tadalafil (1 mg/Kg p.o.) and the combination (Vorinostat, 12.5 mg/Kg+Tadalafil 1 mg/Kg) was studied in Tg2576 mice (14-16 month) after a chronic treatment of 2-5 weeks by using two different behavioral tasks: the Fear Conditioning (FC) and the Morris water maze (MWM) tests. Tg2576 mice express the human 695-aa isoform of APP (hAPP) containing the Swedish double mutation, which favours AP production. See FIG. 2. Results are shown in tables 8 and 9.

The behavioural procedure of FC task involved three phases: habituation, training and testing. During habituation phase, mice were habituated to the conditioning box (context) for 5 min with no stimuli presented. 24 hours later (training phase), mice were placed in the training chamber and allowed to explore for 2 min, afterward, a footshock (0.3 mA) unconditioned stimulus was administered (2 s) and 30 s after mice were returned to their home cage.

Mice were returned to the conditioning chamber 24 h after training and allowed to explore the context for 2 min, during which freezing behavior was recorded (contextual long term memory). Freezing scores were expressed as percentages. The conditioning procedure was carried out in a StartFear system (Panlab S.L., Barcelona, Spain) that allows recording and analysis of the signal generated by the animal movement through a high sensitivity Weight Transducer system. The analogical signal is transmitted to the FREEZING and STARTLE software modulated through the load cell unit for recording purposes and posterior analysis in terms of activity/immobility.

The MWM and the revesal_MWM test was used to evaluate spatial memory as previously described (Ricobaraza et al., 2009; Neuropsychopharmacology, 34, 1721-1732 and Ricobaraza et al., 2011, Frontiers in bioscience, 3, 1375-84). After 3 weeks of treatments, groups of animals underwent spatial reference learning and memory testing in the MWM. The water maze was a circular pool (diameter 1.2 m) filled with water maintained at 20° C. and made opaque by the addition of non-toxic white paint. Mice were trained for three consecutive days (8 trials/day) swimming to a raised platform (visible-platform). No distal visible cues were present during this phase. The same platform location was used for all visible platform sessions and was changed for the invisible-platform training (submerged 1 cm beneath the surface) conducted over 8 consecutive days (4 trials/day) with all visible distal cues present in this phase. In both visible- and hidden-platform versions, mice were placed pseudo-randomly in selected locations, facing toward the wall of the pool to eliminate the potentially confounding contribution of extramaze spatial cues. Each trial was terminated when the mouse reached the platform or after 60 seconds, whichever came first. To test the memory retention, three probe trials were performed at the beginning of 4th, 7th, and the last day of the test (day 9). In the probe trials the platform was removed from the pool, and the percentage of time spent in the quadrant where the platform was previously set was recorded.

After a 4-weeks wash-out period of the drugs, a reversal phase of MWM was carried out. In this phase the platform was placed in the opposite quadrant of the tank and a hidden platform training during 5 consecutive days (four trials per day) was performed. All cues remained in their original positions. Memory retention was analyzed in a probe at day 6.

All trials were monitored by a camera above the center of the pool connected to a SMART-LD program (Panlab S.L., Barcelona, Spain) for subsequent analysis of escape latencies, swimming speed, path length and percent time spent in each quadrant of the pool during probe trials. All experimental procedures were performed blind to groups.

TABLE 8
Percent of freezing in the FC test.

| | FC (% freezing) | p value^{c} |
|---|---|---|
| Vehicle | 44.62 ± 6.9 | |
| V^{a} | 51.17 ± 14.8 | >0.05 |
| T^{b} | 41.38 ± 12.3 | >0.05 |
| V^{a} + T^{b} | 68.36 ± 5.18 | <0.05 |

Mean ± standard desviation value
V, Vorinostat;
T, Tadalafil
^{a}12.5 mg/kg,
^{b}1 mg/kg
^{c}One-way ANOVA followed Sheffè test vs vehicle

TABLE 9
Percent of time on right quadrant in the MWM and
Reversal_MWM.

| | MWM (% of time on | | RMWM (% of time | |
|---|---|---|---|---|
| | right quadrant)^{c} | p value^{d} | on right quadrant)^{e} | p value^{d} |
| Vehicle | 17.1 ± 4.59 | | 12.3 ± 3.93 | |
| V^{a} | 16.3 ± 4.69 | >0.05 | 14.88 ± 5.88 | >0.05 |
| T^{b} | 23.3 ± 7.63 | >0.05 | 21.26 ± 3.40 | >0.05 |
| V^{a} + T^{b} | 35.3 ± 5.35 | <0.05 | 30.61 ± 3.62 | <0.05 |

Mean ± standard desviation value
V. Vorinostat;
T, Tadalafil
^{a}12.5 mg/Kg and
^{b}1 mg/Kg
^{c}Retention phase of MWM test, day 9^{th}
^{d}One-way repeated measures ANOVA followed Sheffè test vs vehicle
^{e}Retention phase of Reversal_MWM test, day 6^{th}.

As shown in Table 9 there is a synergistic in vivo-effect when both compounds are combined as there is only significant effect (p<0.05) with V+T.

Amyloid- and pTau-Based Pathology in Tg2576 Mice

Amyloid and Tau pathology was analyzed in the hippocampus of treated animals. Hippocampus was weighed and homogenized in 8 mass of ice-cold buffer containing a cold lysis buffer with protease inhibitors (0.2 M NaCl, 0.1 M HEPES (4-(2-hydroxyethyl)-1-piperazineethanesulfonic acid), 10% glycerol, 200 mM NaF, 2 mM Na₄P₂O₇, 5 mM EDTA (ethylenediaminetetraacetic acid), 1 mM EGTA (ethylene glycol tetraacetic acid), 2 mM DTT (dithiothreitol), 0.5 mM PMSF (phenylmethylsulfonyl fluoride), 1 mM Na₃VO₄ and Complete Protease Inhibitor Cocktail, Roche Diagnostics), centrifuged at 14,000×g 4° C. for 20 min and the supernatant was aliquoted and stored at −80° C. Total protein concentrations were determined using the BioRad Bradford protein assay (BioRad Laboratories).

Aβ₄₂ levels in hippocampal extracts were measured with the 3D6 antibody (specific for amino acids 1-5 of Aβ; kit from Biosource, USA) using a sensitive sandwich ELISA kit from Biosource (Camarillo, Ca, USA) according to the manufacturer's instructions.

pTau was analyzed by western blot in the hippocampus of treated animals. Protein samples were mixed with Laemmli sample buffer, resolved onto SDS-polyacrylamide gels and transferred to nitrocellulose membrane. The membranes were blocked with 5% milk, 0.05% Tween-20 in PBS (Phosphate-Buffered Saline) or TBS (Tris-Buffered Saline) followed by overnight incubation with the following primary antibodies in the corresponding buffers: mouse monoclonal anti-phosphotau ATB, that recognizes hyperphosphorylated epitopes on Ser202/Thr205 (Pierce Biotechnology Inc., Rockford, 1:1000) and mouse monoclonal anti tau (clone Tau46, Sigma-Aldrich, St Luis, Mo., 1:1000). Following two washes in TBS/Tween20 and one wash in TBS alone, immunolabelled protein bands were detected using HRP-conjugated anti-rabbit or anti-mouse antibodies (diluted 1:5,000 Santa Cruz). Binding was visualised by enhanced chemiluminescence (ECL, GE Healthcare Bioscience, Buckinghamshire, UK) and autoradiographic exposure to Hyperfilm ECL (GE Healthcare Bioscience), using Quantity One™ software v.4.6.3 (Bio-Rad) for quantification.

TABLE 10
Aβ₄₂ levels in hippocampus of treated animals

| | Aβ₄₂ (pg/mg) | p value^{c} |
|---|---|---|
| Vehicle | 22.12 ± 14.65 | |
| V^{a} | 12.81 ± 4.68 | >0.05 |
| T^{b} | 14.89 ± 5.17 | >0.05 |
| V^{a} + T^{b} | 6.60 ± 1.66 | <0.05 |

Mean ± standard desviation value
V, vorinostat;
T, Tadalafil
^{a}12.5 mg/Kg and
^{b}1 mg/Kg
^{c}Student t test vs vehicle

TABLE 11
pTau levels in hippocampus of treated animals

| | pTau/tTau | p value^{c} |
|---|---|---|
| Vehicle | 1.00 ± 0.16 | |
| V^{a} | 0.56 ± 0.09 | >0.05 |
| T^{b} | 0.32 ± 0.03 | <0.05 |
| V^{a} + T^{b} | 0.49 ± 0.08 | <0.05 |

Mean ± standard desviation value
V, Vorinostat;
T, Tadalafil
^{a}12.5 mg/Kg and
^{b}1 mg/Kg
^{c}Student t test vs vehicle

As shown in Tables 10 and 11 there is a synergistic effect on reducing amyloid and tau AD markers when both compounds are combined.

1. A product which is a combination that comprises
a) a phosphodiesterase (PDE) inhibitor compound selected from the group consisting of:
(6R,12aR)-6-(1,3-Benzodioxol-5-yl)-2-methyl-2,3,6,7,12,12a-hexahydropyrazino[1′,2′:1,6]pyrido[3,4-b]indole-1,4-dione (1-30);
5-[2-ethoxy-5-(4-methylpiperazin-1-yl)sulfonyl-phenyl]-1-methyl-3-propyl-6H-pyrazolo[4,3-d]pyrimidin-7-one (1-28);
2-[2-ethoxy-5-(4-ethylpiperazin-1-yl)sulfonyl-phenyl]-5-methyl-7-propyl-1H-imidazo[5,1-f][1,2,4]triazin-4-one (1-29);
3-(1-methyl-7-oxo-3-propyl-6H-pyrazolo[4,3-d]pyrimidin-5-yl)-N-[2-(1-methylpyrrolidin-2-yl)ethyl]-4-propoxy-benzenesulfonamide (1-31);
4-[(3-chloro-4-methoxy-phenyl)methylamino]-2-[(2S)-2-(hydroxymethyl)pyrrolidin-1-yl]-N-(pyrimidin-2-ylmethyl)pyrimidine-5-carboxamide (1-32);
5-ethyl-2-[5-[4-(2-hydroxyethyl)piperazin-1-yl]sulfonyl-2-propoxy-phenyl]-7-propyl-1H-pyrrolo[3,2-d]pyrimidin-4-one (1-33);
5-[2-ethoxy-5-[4-(2-hydroxyethyl)piperazin-1-yl]sulfonyl-phenyl]-1-methyl-3-propyl-6H-pyrazolo[4,3-d]pyrimidin-7-one (1-34);
7-[(3-bromo-4-methoxy-phenyl)methyl]-1-ethyl-8-[[(1R,2R)-2-hydroxycyclopentyl]amino]-3-(2-hydroxyethyl)purine-2,6-dione (1-35);
1-(2-ethoxyethyl)-5-[ethyl(methyl)amino]-7-[(4-methyl-2-pyridyl)amino]-N-methylsulfonyl-pyrazolo[4,3-d]pyrimidine-3-carboxamide (1-36);
2-[(3,4-dimethoxyphenyl)methyl]-7-[(1R)-1-[(1R)-1-hydroxyethyl]-4-phenyl-butyl]-5-methyl-1H-imidazo[5,1-f][1,2,4]triazin-4-one (1-01);
4-(azetidin-1-yl)-7-methyl-5-[1-methyl-5-[5-(trifluoromethyl)-2-pyridyl]pyrazol-4-yl]imidazo[5,1-f][1,2,4]triazine (1-02);
3-(8-methoxy-1-methyl-2-oxo-7-phenyl-3H-1,4-benzodiazepin-5-yl)benzamide (1-03);
6-[4-(1-cyclohexyltetrazol-5-yl)butoxy]-3,4-dihydro-1H-quinolin-2-one (1-04);
6-methyl-2-oxo-5-(4-pyridyl)-1H-pyridine-3-carbonitrile (1-05);
4-methyl-5-(4-methylsulfanylbenzoyl)-1,3-dihydroimidazol-2-one (1-06);
3-amino-5-(4-pyridyl)-1H-pyridin-2-one (1-07);
5-imidazo[1,2-a]pyridin-6-yl-6-methyl-2-oxo-1H-pyridine-3-carbonitrile (1-08);
1-cyclopropyl-1-[(1R,2R)-2-hydroxycyclohexyl]-3-[3-[(2-oxo-1H-quinolin-6-yl)oxy]propyl]urea (1-09);
4-[3-(cyclopentoxy)-4-methoxy-phenyl]pyrrolidin-2-one (1-10);
2-[(E)-[3-(cyclopentoxy)-4-methoxy-phenyl]methyleneamino]oxy-1-(2,6-dimethylmorpholin-4-yl)ethanone (1-11);
5-[5-[(2,4-dimethylthiazol-5-yl)methyl]-1,3,4-oxadiazol-2-yl]-1-ethyl-N-tetrahydropyran-4-yl-pyrazolo[3,4-b]pyridin-4-amine (1-12);
6-[3-(dimethylcarbamoyl)phenyl]sulfonyl-4-(3-methoxyanilino)-8-methyl-quinoline-3-carboxamide (1-13);
N-[2-[(1S)-1-(3-ethoxy-4-methoxy-phenyl)-2-methylsulfonyl-ethyl]-1,3-dioxo-isoindolin-4-yl]acetamide (1-14);
(1R,2R)-2-[4-[3-[3-(cyclopropylcarbamoyl)-4-oxo-1,8-naphthyridin-1-yl]phenyl]-2-fluoro-phenyl]cyclopropanecarboxylicacid (1-15);
3-(cyclopropylmethoxy)-N-(3,5-dichloro-4-pyridyl)-4-(difluoromethoxy)benzamide (1-16);
4-[(1-hydroxy-3H-2,1-benzoxaborol-5-yl)oxy]phthalonitrile (1-17);
4-[(1-hydroxy-3H-2,1-benzoxaborol-5-yl)oxy]benzonitrile (1-18);
4-cyano-4-[3-(cyclopentoxy)-4-methoxy-phenyl]cyclohexanecarboxylicacid (1-19);
(1Z)-1-[(3,4-diethoxyphenyl)methylene]-6,7-diethoxy-3,4-dihydro-2H-isoquinoline (1-20);
N-(3,5-dichloro-4-pyridyl)-2-[1-[(4-fluorophenyl)methyl]pyrrolo[2,3-b]pyridin-3-yl]-2-oxo-acetamide (1-21);
N-(3,5-dichloro-1-oxido-pyridin-1-ium-4-yl)-6-(difluoromethoxy)benzofuro[3,2-c]pyridine-9-carboxamide (1-22);
6-[2-(3,4-diethoxyphenyl)thiazol-4-yl]pyridine-2-carboxylicacid (1-23);
methyl4-[[3-[6,7-dimethoxy-2-(methylamino)quinazolin-4-yl]phenyl]carbamoyl]benzoate (1-24);
2-[(2E)-9,10-dimethoxy-4-oxo-2-(2,4,6-trimethylphenyl)imino-6,7-dihydropyrimido[6,1-a]isoquinolin-3-yl]ethylurea (1-25);
3-benzyl-5-phenyl-1H-pyrazolo[4,3-c][1,8]naphthyridin-4-one (1-26);
(3 S,5 S)-5-[3-(cyclopentoxy)-4-methoxy-phenyl]-3-(m-tolylmethyl)piperidin-2-one (1-27);
3-phenyl-2-thioxo-1H-quinazolin-4-one (1-37);
3-[[(2R)-4-(thiazol-2-ylmethyl)morpholin-2-yl]methyl]-5-(trifluoromethyl)triazolo[4,5-d]pyrimidin-7-amine (1-38);
6-[(3 S,4 S)-4-methyl-1-(pyrimidin-2-ylmethyl)pyrrolidin-3-yl]-1-tetrahydropyran-4-yl-5H-pyrazolo[3,4-d]pyrimidin-4-one (1-39);
2-[[4-[1-methyl-4-(4-pyridyl)pyrazol-3-yl]phenoxy]methyl]quinoline (1-40);
RG7203 (1-41); TAK-063 (1-42); OMS824 (1-43); FRM-6308 (1-44);
BI409306 (1-45); N-cyclopropyl-4-oxo-1-[3-[2-(1-oxo-3-pyridyl)ethynyl]-phenyl]-1,8-naphthyridine-3-carboxamide (1-46); and
1-[4-[3-[4-(1H-benzimidazole-2-carbonyl)phenoxy]pyrazin-2-yl]-1-piperidyl]ethanone (1-47);
or a pharmaceutically or veterinary acceptable salt thereof, or any stereoisomer thereof, either of the compound of formula (1-01) to (1-47) or of any of its pharmaceutically or veterinary acceptable salts, and
b) a histone deacetylase (HDAC) inhibitor compound selected from the group consisting of:
8-(hydroxyamino)-8-oxo-N-phenyl-octanamide (2-01);
(1 S,4 S,7Z,10 S,16E,21R)-7-ethylidene-4,21-diisopropyl-2-oxa-12,13-dithia-(E)-3-[4-[[2-(2-methyl-1H-indol-3-yl)ethylamino]methyl]phenyl]prop-2-enehydroxamic acid (2-03);
(E)-3-[3-(phenylsulfamoyl)phenyl]prop-2-enehydroxamic acid (2-07);
2-propylpentanoic acid (2-17);
5,8,20,23-tetrazabicyclo[8.7.6]tricos-16-ene-3,6,9,19,22-pentone (2-02);
3-pyridylmethyl N-[[4-[(2-aminophenyl)carbamoyl]phenyl]methyl]carbamate (2-04);
N-(2-aminophenyl)-4-[[[4-(3-pyridyl)pyrimidin-2-yl]amino]methyl]benzamide (2-05);
N-[7-(hydroxyamino)-7-oxo-heptyl]-2-(N-phenylanilino)pyrimidine-5-carboxamide (2-06);
(E)-3-[1-[4-[(dimethylamino)methyl]phenyl]sulfonylpyrrol-3-yl]prop-2-enehydroxamic acid (2-08);
[6-(diethylaminomethyl)-2-naphthyl]methyl N-[4-(hydroxycarbamoyl)phenyl]-carbamate (2-09);
(E)-3-[2-butyl-1-[2-(diethylamino)ethyl]benzimidazol-5-yl]prop-2-enehydroxamic acid (2-10);
3-[(dimethylamino)methyl]-N-[2-[4-(hydroxycarbamoyl)phenoxy]ethyl]benzofuran-2-carboxamide (2-11);
(E)-N-(2-aminophenyl)-3-[1-[4-(1-methylpyrazol-4-yl)phenyl]sulfonylpyrrol-3-yl]prop-2-enamide (2-12);
(2S)—N-[4-(hydroxycarbamoyl)phenyl]-3-methyl-2-phenyl-butanamide (2-13);
(E)-N-[3-(dimethylamino)propyl]-8-(hydroxyamino)-2-(1-naphthyloxymethyl)-8-oxo-oct-2-enamide (2-14);
4-[(2-methyl-3,4-dihydro-1H-pyrido[4,3-b]indol-5-yl)methyl]benzenecarbohydroxamic acid (2-15)
sodium; 4-phenylbutanoate (2-16);
N-(2-amino-5-fluoro-phenyl)-4-[[[(E)-3-(3-pyridyl)prop-2-enoyl]amino]methyl]benzamide (2-18);
2-[4-[[(1-methylindol-3-yl)methylamino]methyl]-1-piperidyl]pyrimidine-5-carbohydroxamic acid (2-19);
cyclopentyl (2S)-2-[[4-[[8-(hydroxyamino)-8-oxo-octanoyl]amino]phenyl]-methylamino]-2-phenyl-acetate (2-20);
2-[(1R,5S)-6-[(6-fluoro-2-quinolyl)methylamino]-3-bicyclo[3.1.0]hexanyl]pyrimidine-5-carbohydroxamic acid (2-21);
N-[6-(2-aminoanilino)-6-oxo-hexyl]-4-methyl-benzamide (2-22);
4-acetamido-N-(2-aminophenyl)benzamide (2-23);
ACY-241 (2-24); OCID-4681 (2-25) and FRM-0334 (2-26)
or a pharmaceutically or veterinary acceptable salt thereof, or any stereoisomer thereof, either of the compound of formula (2-01) to (2-26) or of any of its pharmaceutically or veterinary acceptable salts.

2. The product according to claim 1, wherein the PDE inhibitor compound is a selective PDE5 inhibitor.

3. The product according to claim 2, wherein the selective PDE5 inhibitor compound is selected from the group consisting of compounds of formula (1-30), (1-28) and (1-29).

4. The product according to claim 1, wherein the HDAC inhibitor compound is a Pan-HDAC inhibitor selected from the group consisting of compounds of formula (2-01), (2-03), (2-07) and (2-17) or class-I inhibitor compound of formula (2-02).

5. The product according to claim 4, wherein the Pan-HDAC inhibitor is compound of formula (2-01).

6. The product according to claim 5, wherein the PDE inhibitor compound is a selective PDE5 inhibitor compound selected from the group consisting of compounds of formula (1-30), (1-28) and (1-29); and the Pan-HDAC inhibitor is compound of formula (2-01).

7. The product according to claim 6, wherein the selective PDE5 inhibitor is compound of formula (1-30) and the Pan-HDAC inhibitor is compound of formula (2-01).

8. A product which is a single pharmaceutical or veterinary composition which comprises a therapeutically effective amount of:
a) a PDE inhibitor compound selected from the group consisting of compounds of formula (1-01) to (1-47) as defined in claim 1, or a pharmaceutically or veterinary acceptable salt thereof, or any stereoisomer either of the compound or of any of its pharmaceutically or veterinary acceptable salts; and of
b) a HDAC inhibitor compound, of formula (2-01) to (2-26) as defined in claim 1, or a pharmaceutically or veterinary acceptable salt thereof, or any stereoisomer either of the compound or of any of its pharmaceutically or veterinary acceptable salts,
together with one or more pharmaceutically or veterinary acceptable excipients or carriers.

9. A product which is a package or kit of parts comprising:
a) i) a pharmaceutical or veterinary composition which comprises a therapeutically effective amount of a PDE inhibitor compound selected from the group consisting of compounds of formula (1-01) to (1-47) as defined in claim 1, or a pharmaceutically or veterinary acceptable salt thereof, or any stereoisomer thereof, either of the compound of formula (1-01) to (1-47) or of any of its pharmaceutically or veterinary acceptable salts, together with one or more pharmaceutically or veterinary acceptable excipients or carriers; and
ii) instructions for simultaneous, concurrent, separate or sequential use of the composition i) in combination with a pharmaceutical or veterinary composition which comprises a therapeutically effective amount of a HDAC inhibitor compound of formula (2-01) to (2-26) as defined in claim 1, or a pharmaceutically or veterinary acceptable salt thereof, or any stereoisomer thereof, either of the compound of formula (2-01) to (2-26) or of any of its pharmaceutically or veterinary acceptable salts, together with one or more pharmaceutically or veterinary acceptable excipients or carriers; or alternatively,
b) i′) a pharmaceutical or veterinary composition which comprises a therapeutically effective amount of a HDAC inhibitor compound of formula (2-01) to (2-26) as defined in claim 1, or a pharmaceutically or veterinary acceptable salt thereof, or any stereoisomer thereof, either of the compound of formula (2-01) to (2-26) or of any of its pharmaceutically or veterinary acceptable salts, together with one or more pharmaceutically or veterinary acceptable excipients or carriers; and
ii′) instructions for simultaneous, concurrent, separate or sequential use of the composition i′) in combination with a pharmaceutical or veterinary composition which comprises a therapeutically effective amount of a PDE inhibitor compound selected from the group consisting of compounds of formula (1-01) to (1-47) as defined in claim 1, or a pharmaceutically or veterinary acceptable salt thereof, or any stereoisomer thereof, either of the compound of formula (1-01) to (1-47) or of any of its pharmaceutically or veterinary acceptable salts, together with one or more pharmaceutically or veterinary acceptable excipients or carriers.

10. A product which is a package or kit of parts comprising:
a) a first pharmaceutical or veterinary composition which comprises a therapeutically effective amount of a PDE inhibitor compound selected from the group consisting of compounds of formula (1-01) to (1-47) as defined in claim 1, or a pharmaceutically or veterinary acceptable salt thereof, or any stereoisomer thereof, either of the compound of formula (1-01) to (1-47) or of any of its pharmaceutically or veterinary acceptable salts, together with one or more pharmaceutically or veterinary acceptable excipients or carriers; and
b) a second pharmaceutical or veterinary composition which comprises a therapeutically effective amount of a HDAC inhibitor compound of formula (2-01) to (2-26) as defined in claim 1, or a pharmaceutically or veterinary acceptable salt thereof, or any stereoisomer thereof, either of the compound of formula (2-01) to (2-26) or of any of its pharmaceutically or veterinary acceptable salts, together with one or more pharmaceutically or veterinary acceptable excipients or carriers;
wherein compositions a) and b) are separate compositions.

11. A method for the treatment and/or prevention of a neurological disorder coursing with a cognition deficit or impairment, or a neurodegenerative disease, which comprises administering to a mammal subject in need thereof, including a human subject, a therapeutically effective amount of a product which is a combination that comprises a phosphodiesterase (PDE) inhibitor compound and a histone deacetylase (HDAC) inhibitor compound as defined in claim 1 and one or more pharmaceutical acceptable excipients or carriers.

12. The method according to claim 11, wherein the neurodegenerative disease is a neurodegenerative disease coursing with a cognition deficit or impairment selected from Alzheimer's disease, Parkinson's disease, Huntington's disease, vascular dementia (uncomplicated, with delirium, with delusions or with depressed mood), mild cognitive impairment and age-associated cognition impairment.

13. The method according to claim 12, wherein the disease is Alzheimer's disease.

14. (canceled)

15. (canceled)

16. The product according to claim 3, wherein the HDAC inhibitor compound is a Pan-HDAC inhibitor selected from the group consisting of compounds of formula (2-01), (2-03), (2-07) and (2-17) or class-I inhibitor compound of formula (2-02).

17. The method according to claim 11, wherein the treatment comprises the simultaneous, concurrent, separate or sequential administration of the phosphodiesterase (PDE) inhibitor compound and the histone deacetylase (HDAC) inhibitor compound.

18. A method for the treatment and/or prevention of a neurological disorder coursing with a cognition deficit or impairment, or a neurodegenerative disease, which comprises administering to a mammal subject in need thereof, including a human subject, a therapeutically effective amount of a product as defined in claim 5 and one or more pharmaceutical acceptable excipients or carriers.

19. The method according to claim 18, wherein the treatment comprises the simultaneous, concurrent, separate or sequential administration of the phosphodiesterase (PDE) inhibitor compound and the histone deacetylase (HDAC) inhibitor compound.

20. A method for the treatment and/or prevention of a neurological disorder coursing with a cognition deficit or impairment, or a neurodegenerative disease, which comprises administering to a mammal subject in need thereof, including a human subject, a therapeutically effective amount of a product as defined in claim 8.

21. A method for the treatment and/or prevention of a neurological disorder coursing with a cognition deficit or impairment, or a neurodegenerative disease, which comprises administering to a mammal subject in need thereof, including a human subject, a therapeutically effective amount of a product as defined in claim 10.

22. The method according to claim 21, wherein the treatment comprises the simultaneous, concurrent, separate or sequential administration of the phosphodiesterase (PDE) inhibitor compound and the histone deacetylase (HDAC) inhibitor compound.
